(19) United States
(12) Patent Application Publication
(10) Pub. No.: US 2025/0381184 A1
(43) Pub. Date: Dec. 18, 2025

(54) PHARMACEUTICAL COMBINATION COMPRISING AN ANTI-CD3 ANTIBODY AND A CXCR3 ANTAGONIST

(71) Applicant: Idorsia Pharmaceuticals Ltd, Allschwil (CH)

(72) Inventors: Monika BAYER, Frankfurt am Main (DE); Urs CHRISTEN, Frankfurt am Main (DE); Edith HINTERMANN, Frankfurt am Main (DE); Marianne MARTINIC, Allschwil (CH); Ulrich MENTZEL, Allschwil (CH); Laetitia POUZOL, Allschwil (CH); Camilla TONDELLO, Neuherberg (DE)

(21) Appl. No.: 19/109,802

(22) PCT Filed: Sep. 7, 2023
(86) PCT No.: PCT/EP2023/074546

§ 371 Date: Mar. 7, 2025

(30) Foreign Application Priority Data
Sep. 9, 2022 (WO) PCT/EP2022/075099

(51) Int. Cl. A61K 31/506; A61K 9/00; A61K 39/00; A61K 39/395; A61P 3/10

(52) U.S. Cl. CPC A61K 31/506; A61K 9/0019; A61K 9/0053; A61K 39/3955; A61P 3/10; A61K 2039/505; A61K 2039/54; A61K 2039/545

(57) ABSTRACT

The present invention relates to a pharmaceutical combination comprising a first active ingredient which is the CXCR3 antagonist 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, and a second active ingredient which is an anti-CD3 monoclonal antibody (mAb); and to the use of the pharmaceutical combination in the prevention, prophylaxis and/or treatment of (auto-)immune/inflammatory mediated disorders, including type 1 diabetes (T1D) (especially autoimmune T1D), multiple sclerosis, organ transplant rejection (especially renal and heart allograft rejection), thyroid eye disease, rheumatoid arthritis, ulcerative colitis, crohn's disease, celiac disease, atherosclerosis, psoriasis, lung inflammation, and psoriatic arthritis.

The present invention relates to a pharmaceutical combination comprising a first active ingredient which is the CXCR3 antagonist 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone (hereinafter also referred to as “COMPOUND”), or a pharmaceutically acceptable salt thereof, and a second active ingredient which is an anti-CD3 monoclonal antibody (mAb); and to the use of the pharmaceutical combination in the prevention, prophylaxis and/or treatment of (auto-) immune/inflammatory mediated disorders, including type 1 diabetes (T1D) (especially autoimmune T1D), multiple sclerosis, organ transplant rejection (especially renal and heart allograft rejection), thyroid eye disease, rheumatoid arthritis, ulcerative colitis, crohn's disease, celiac disease, atherosclerosis, psoriasis, lung inflammation, and psoriatic arthritis.

Chemokine receptors are a group of G-protein coupled receptors (GPCRs) that bind peptidic chemokine ligands with high affinity. The predominant function of chemokine receptors is to guide leukocyte trafficking to lymphoid organs and tissues under resting conditions as well as during inflammation, but a role for certain chemokine receptors on non-hematopoietic cells and their progenitors has also been recognized.

The chemokine receptor CXCR3 is a G-protein coupled receptor binding to the inflammatory chemokines CXCL9 (initially called MIG, monokine induced by interferon-γ [INF-γ]), CXCL10 (IP-10, INF-γ-inducible protein 10), and CXCL11 (I-TAC, INF-γ-inducible T cell α chemo-attractant). CXCR3 is mainly expressed on activated T helper type 1 (Th1) lymphocytes, but is also present on natural killer cells, macrophages, dendritic cells and a subset of B lymphocytes. The three CXCR3 ligands are expressed mainly under inflammatory conditions, expression in healthy tissue is very low. Cells that can express CXCR3 ligands, for instance after exposure to inflammatory cytokines such as interferon-γ or TNF-α include diverse stromal cells such as endothelial cells, fibroblasts, epithelial cells, keratinocytes but also includes hematopoietic cells such as macrophages and monocytes. The interaction of CXCR3 and its ligands (henceforth referred to as the CXCR3 axis) is involved in guiding receptor bearing cells to specific locations in the body, particularly to sites of inflammation, immune injury and immune dysfunction and is also associated with tissue damage, the induction of apoptosis, cell growth, and angiostasis. CXCR3 and its ligands are upregulated and highly expressed in diverse pathological situations including autoimmune disorders, inflammation, infection, transplant rejection, fibrosis, neurodegeneration, and cancer. As discussed in more detail in WO 2022/162017/PCT/EP2022/051786, the CXCR axis is involved in a variety of disease like rheumatoid arthritis, multiple sclerosis, Crohn's disease, ulcerative colitis, systemic lupus erythematosus, lupus nephritis, sarcoidosis, systemic sclerosis, psoriasis, psoriatic arthritis, interstitial cystitis, celiac disease, myasthenia gravis, type 1 diabetes, vitiligo, uveitis, dry eye disease, transplant rejection, acute and/or chronic graft versus host disease, acute lung injury, acute respiratory distress syndrome, asthma, chronic obstructive pulmonary disorder, atherosclerosis, myocarditis, influenza, cerebral malaria, liver cirrhosis, Alzheimer's disease, neurodegeneration, Huntington's chorea, neuromyelitis optica, chronic inflammatory demyelinating polyneuropathy, Guillain-Barré syndrome, brain tumor, colon cancer, breast cancer, and/or metastatic spread of cancer. The CXCR3 axis is further linked to intracerebral hemorrhage (ICH). Landreneau et al. reported that in a prospective cohort study completed by 115 patients an elevated serum CXCL10 concentration 24 and 72 hours after ICH was associated with worse functional outcome at 90 days after ICH onset (Landreneau et al., Annals of Clinical and Translational Neurology 2018; 5(8): 962-970). Furthermore, Leung et al. investigated the outcome of experimental intracerebral hemorrhage in CXCR3 Knock-Out mice and concluded: “CXCR3 Knock-Out mice had better motor functions especially in the first week after ICH. The degree of demyelination of the CXCR3 Knock-out mice was less severe compared to that of the Wild-Type mice” (Leung et al., 28th Annual Scientific Meeting of The Hong Kong Neurosurgical Society: Updates on Traumatic Brain Injury and Neurocritical Care, Virtual Meeting, Hong Kong, 26-27 Nov. 2021, abstract, http://hdl.handle.net/10722/309045).

T1D is characterized by hyperglycemia and by the destruction of insulin-producing B-cells. It is classified in two subtypes: the common (85-90%), strongly HLA-associated, immune mediated form, with presence of autoantibodies against one or more of the following autoantigens: islet cell (ICA), GAD, insulin, IA-2, and ZnT8; and the less common, strongly inherited but not HLA-associated, idiopathic form, with no evidence of β-cell autoimmunity. The destruction of pancreatic islet β-cells results in insulin deficiency which leads to life-threatening glucose dysregulation. Hyperglycemia is responsible for the development of debilitating microvascular complications associated with a significant higher risk of mortality as compared to the unaffected population (Laing SP, et al. Stroke 2003a 34(2):418-21; Laing SP, et al. Diabetologia 2003b 46(6):760-5). While T1D can be controlled by subcutaneous administration of exogenous insulin, there is no permanent cure, therefore a lifelong management is required. The autoimmune process associated with T1D is believed to start years prior to clinical diagnosis and involves humoral and cellular immune responses, as evidenced by the emergence of anti-islet autoantibodies. At diagnosis, most individuals with T1D are believed to retain some level of functioning β cells, as indicated by the presence of circulating C-peptide, a byproduct of endogenous insulin processing and further supported by histological analysis of patients with recently diagnosed T1D who died from causes unrelated to diabetes. The preservation of these functional β cells is associated with fewer clinical complications and maintaining or even enhancing a functional β cell compartment is of high medical and pharmacological interest. T1D is a bona fide autoimmune disease as shown by the presence of B cell secreted autoantibodies, even in a pre-symptomatic stage of the disease, detection of autoreactive T-cell receptors on both CD4+ and CD8+ T cell subtypes in the pancreata from patients with recent onset T1D, and the fact that genetic risk factors for T1D are enriched for immune related genes (Katsarou et al, Nat Rev Dis Primers. 2017, 3:17016).

First evidence that inhibiting the autoimmune response in recently diagnosed patients with T1D could lead to a preservation of β cell compartment, associated with improved metabolic control, came from a trial using the calcineurin inhibitor, cyclosporine (Stiller CR, et al. Science 1984 223(4643):1362-7). However, the benefits of this treatment were outweighed by its safety liabilities, which prevented this drug from being used in the clinics. So far, no general immunosuppressive drug has been approved for T1D, due to overall unfavorable safety profiles in a mainly pediatric target population (Chatenoud L. Diabetologia 2019 62(4):578-81).

New strategies have emerged to induce immune tolerance against self-antigens, using antigen-based immunotherapies and immunomodulatory agents to avoid chronic immunosuppression (Jacobsen LM, et al. Curr Diab Rep 2018 18(10):90). Several clinical trials with agents aiming to prevent or delay the development of T1D have demonstrated only a transient and modest efficacy, preventing the regulatory approval of these therapies (Chatenoud L. Diabetologia 2019 62(4):578-81). As an example, while the continuous administration of Abatacept (CTLA4lg) slowed progression of the disease over two years, the treatment only delayed but did not prevent the decline in β cell function and followed a kinetic parallel to that of placebo (Orban T, et al. Lancet 2011 378(9789):412-9).

Successful attempts to stop the progression of T1D with more specific immunomodulating agents were first reported in a pre-clinical model of T1D (non-obese diabetes (NOD) mouse model) by using a mouse specific anti-CD3 mAb (clone 145-2C1) (Chatenoud L, et al. Proc Natl Acad Sci U S A 1994 91(1):123-7). This therapeutic anti-CD3 mAb binds to the epsilon chain of the CD3/TCR complex found on T lymphocytes. Specifically, Chatenoud et al. demonstrated that a 5-day treatment with anti-CD3 mAb (5 μg/day), administrated intravenously, induced rapid, long-lasting remission from T1D in diabetic NOD mice. Treatment with anti-CD3 mAb prevented an immune response towards syngeneic pancreatic islet grafts but did not impair normal rejection observed with skin allografts (Chatenoud L, et al. Proc Natl Acad Sci U S A 1994 91(1): 123-7). Therefore, efficacy of the transient targeting of the CD3/T-cell receptor with anti-CD3 mAb treatment was proposed to be due to a restoration of immune tolerance against self-antigens via preferential killing of activated effector T cells and/or expansion of regulatory T cells (Chatenoud L, et al. Proc Natl Acad Sci USA 1994 91(1):123-7). This discovery led to the initiation of several preclinical studies demonstrating that treatment with intravenous administration of anti-CD3 mAb could be of potential use, alone, or in combination, for tolerance induction in autoimmune diseases and other immune-mediated pathologies (Chatenoud L and Waldmann H. Rev Diabet Stud 2012 9(4):372-81), providing beneficial effects in experimental animal disease models modelling T1D (Chatenoud L, et al. Proc Natl Acad Sci USA 1994 91(1):123-7), multiple sclerosis (Tran GT, et al. Int Immunol 2001 13(9):1109-20), inflammatory bowel disease (IBD) (Ludviksson BR, et al. J Immunol 1997 159(7):3622-8), rheumatoid arthritis (Hughes C, et al. J Immunol 1994 153(7):3319-25), graft versus host disease (Blazar BR, et al. J Immunol 1994 152(7):3665-74), organ transplant rejection (Nicolls MR, et al. Transplantation 1993 55(3):459-68), and atherosclerosis (Kita T, et al. Cardiovasc Res 2014 102(1):107-17). Of note, Kuhn et al, have also reviewed efficacy of anti-CD3 mAb administered orally or intranasally in experimental animal autoimmune models (Kuhn C and Weiner HL. Immunotherapy 2016 8(8):889-906).

The first anti-CD3 mAb, marketed under the trade name muromonab (Orthoclone OKT3) is a murine IgG2a antibody approved by the US food and drug administration (FDA) in 1986 for inhibiting acute allograft rejection in solid-organ transplantation (Hooks MA, et al. Pharmacotherapy 1991 11(1):26-37). However, muromonab administered once a day for several consecutive days, elicits a high titer of anti-mouse antibodies in humans and is a potent mitogen, inducing massive amounts of cytokines and leading to a wide spectrum of side effects including severe side effects such as encephalopathy, meningitis, graft thrombosis and renal insufficiency (Sgro C. Toxicology 1995 105(1):23-9). As the immunogenicity of muromonab is caused by its rodent origin, next generation of anti-CD3 mAb were humanized and rendered less mitogenic by introducing mutations to reduce affinity of the antibodies to Fc receptors (FcR) on antigen presenting cells. So far, four humanized anti-human CD3 mAb, including humanized versions of rodent anti-human CD3 mAb (otelixizumab, teplizumab, visilizumab) and fully human mAb (foralumab), have been investigated in human clinical trials (Kuhn C and Weiner HL. Immunotherapy 2016 8(8):889-906). To overcome the undesirable effects observed with muromonab, all four antibodies have reduced or no FcR binding affinity.

While visilizumab and foralumab were mostly studied in inflammatory bowel disease (Dean Y, et al. Swiss Med Wkly 2012 142:w13711), otelixizumab and teplizumab have been evaluated in several clinical trials and have independently shown efficacy in patients with recent onset T1D (Chatenoud L. Diabetologia 2019 62(4):578-81).

Otelixizumab, also known as ChAglyCD3, TRX4, GSK2136525, is a chimeric mAb derived from the rat antibody YTH12.5 and is a humanized IgG1 antibody bearing a single mutation in the γ1 Fc portion. The potential efficacy of otelixizumab in the treatment of T1D has been widely investigated in human clinical trials (Guglielmi C, et al. Expert Opinion on Biological Therapy 2016 16(6):841-6). Specifically, intravenous treatment with otelixizumab was tested in a large randomized, placebo-controlled, phase II clinical study in patients with new-onset T1D. A total of 48-64 mg of otelixizumab over 6 consecutive days (8 mg/day; the first nine patients received 24 mg on day 1 followed by 8 mg/day) demonstrated efficacy as shown by maintenance of C-peptide levels and reduced insulin requirements. Further analysis demonstrated that the efficacy was more pronounced among patients that had higher residual β cell function and a younger age at baseline (Keymeulen B, et al. N Engl J Med 2005 352(25):2598-608; Keymeulen B, et al. Diabetologia 2010 53(4):614-23). However, administration of otelixizumab was associated with an increased rate of symptomatic Epstein-Barr virus (EBV) reactivation and with moderate “flu-like” syndrome (Keymeulen B, et al. N Engl J Med 2005 352(25):2598-608). A follow-up phase III study was designed to assess whether a lower dose of otelixizumab (3.1 mg total dose over 8 days) could be efficacious while reducing the above-mentioned side effects. However, treatment with otelixumab at this dose while being very well tolerated did not meet primary endpoints.

Teplizumab, also named hOKT3γ1 (Ala-Ala), MGA031, PRV-031, teplizumab-mzwv, and Tzield, is a humanized IgG1 antibody, engineered to have two point mutations in its Fc portion for FcR non-binding properties. Teplizumab is currently in development for the treatment of patients with recent onset type 1 diabetes and in individuals at risk for developing T1D (Herold KC, et al. N Engl J Med 2019 381(7):603-13). Efficacy of teplizumab was assessed in a phase I-II clinical trial in patients with recent onset T1D (Herold KC, et al. N Engl J Med 2002 346(22):1692-8; Herold KC, et al. Diabetes 200554(6):1763-9). Of 21 subjects with recent onset T1D (diagnosis within 6 weeks), treated with teplizumab intravenously for 14 days, 15 had maintained or improved C-peptide responses after 1 year compared to 4 out 19 control subjects. In addition, the study demonstrated that use of insulin was reduced, and glycated haemoglobin levels were also improved in the drug-treated cohort. In addition, teplizumab was well tolerated. The AbATE trial, a randomized, open-label phase 2 study, subsequently showed a positive impact of the anti-CD3 monoclonal antibody teplizumab on preservation of insulin secretion in patients newly diagnosed with T1D (Herold KC, et al. Diabetes 2013 62(11):3766-74). Drug-treated patients were treated intravenously with teplizumab once a day for 14 days, following an uptitration regimen (day 1, 51 μg/m²; day 2, 103 μg/m²; day 3, 206 μg/m²; day 4, 413 μg/m²; days 5-14, 826 μg/m² body surface with a median cumulative dose of 11.6 mg). After one year, patients could receive a second treatment cycle. Two years after initiation of the study, patients treated with teplizumab had a mean C-peptide area under the curve (AUC) 75% higher as compared to controls (Herold KC, et al. Diabetes 2013 62(11):3766-74). While the clinical benefit afforded by teplizumab administration was clinically meaningful and valuable, it was only transient and not all patients responded to the treatment (Herold KC, et al. Diabetes 2013 62(11):3766-74; Perdigoto AL, et al. Diabetologia 2019 62(4):655-64). Approximately 45% of the drug-treated subjects were classified as reponders, defined as patients who lost <40% of baseline C-peptide. While in these patients the effects of teplizumab were robust and durable, in the non-responders, the effects of teplizumab were modest. Of note, non-responders had increased numbers of blood IFN-γ-producing CD8+ T cells at baseline compared to responders (Herold KC, et al. Diabetes 2013 62(11):3766-74). In November 2022, teplizumab-mzwv was approved by the U.S. Food and Drug Administration for delaying the onset of Stage 3 type 1 diabetes (T1D) in adults and pediatric patients aged 8 years and older with Stage 2 T1D. According to the Prescribing Information (label), teplizumab-mzwv is to be administered by intravenous infusion (over a minimum of 30 minutes), using a body surface area-based dosing, once daily for 14 consecutive days as follows: day 1, 65 μg/m²; day 2, 125 μg/m²; day 3, 250 μg/m²; day 4, 500 μg/m²; days 5 through 14, 1030 μg/m².

Clinically, anti-CD3 mAbs have also demonstrated beneficial effects in several indications such as organ transplant rejection, thyroid eye disease, rheumatoid arthritis, ulcerative colitis, crohn's disease, psoriasis, and psoriatic arthritis (Dean Y, et al. Swiss Med Wkly 2012 142:w13711).

Visilizumab, also named Nuvion and HuM291, is a humanized IgG2 antibody, engineered to have two point mutations in its Fc portion for FcR non-binding properties. Efficacy and safety of visilizumab was assessed in a phase I trial in patients with severe corticosteroid-refractory ulcerative colitis. The original dose of 15 μg/kg/day that had to be reduced to 10 μg/kg/day to decrease the prolonged lymphopenia, gave promising results with 84% of the patients demonstrating a clinical response (Plevy S, et al. Gastroenterology 2007 133(5), 1414-1422). However, in follow-up trials, treatment with visilizumab was stopped prematurely due to safety and efficacy concerns, including cytokine release syndrome and increased rate of infection, probably resulting from a stronger CD3 signaling activation (Sandborn WJ, et al. Gut 2010 59(11), 1485-1492; Dean Y, et al. Swiss Med. 2012 Wkly 142, w13711).

Foralumab, also named NI-0401, is the only fully human IgG1 monoclonal anti-CD3 antibody, currently being developed for the treatment of Crohn's and neurodegenerative diseases, such as secondary progressive MS. Efficacy and safety of foralumab has been assessed in a Phase I/II clinical trial in patients with moderate to severe active Crohn's disease (Van der woude CJ, et al. Inflamm. Bowel Dis 2010 16(10), 1708-1716). The patients treated with foralumab (1 mg intravenously, daily for 5 days) demonstrated reduced Crohn's disease endoscopy index of severity at week 6 compared to placebo group. In addition, in a pilot trial, nasal administration of foralumab (100 μg/day for 10 days) has been assessed in mild to moderate non-hospitalized COVID-19 patients. Subjects treated with foralumab demonstrated reduced serum IL-6, C-reactive protein, and more rapid clearance of lung infiltrates (Moreira TG, et al. Front Immunol 2021 12, 709861). Moreover, in a patient with secondary progressive multiple sclerosis, treated for six months with intranasal foralumab, inhibition of microglial activation, downregulation of pro-inflammatory cytokines, and stabilization of disease was observed (Tiziana, press release Mar. 10, 2022: “Tiziana Announces Positive Clinical Data from A Secondary Progressive Multiple Sclerosis Patient Treated for Six Months with Intranasally Administered Foralumab, A Fully Human Anti-CD3 Monoclonal Antibody”, https://ir.tizianalifesciences.com/news-releases/news-release-details/tiziana-announces-positive-clinical-data-secondary-progressive). Tiziana announced to investigate intranasal Foralumab in additional diseases like Alzheimer's disease, long COVID, early onset type 1 diabetes melitus, amyotrophic lateral sclerosis and intracerebral hemorrhage.

T1D is a pathophysiologically complex disease and the clinical therapeutic benefits of anti-CD3 mAb wane over time. For example, there may be individual factors that lead to the escape from the efficacy of immune therapy, including anti-CD3 therapy, such as inflammatory mediators.

Inflammatory mediators are involved in the pathogenesis of T1D. Specifically, the pathological role of the CXCR3 axis in T1D is well-known from the literature. CXCR3 is a cell surface chemokine receptor expressed on adaptive and innate immune cells. It is found on a subset of naïve and activated CD4+ and CD8+ T lymphocytes as well as on subsets of regulatory T cells, B-cells, natural killer cells, myeloid cells, and plasmacytoid dendritic cells (Groom JR, Luster AD.; Immunol Cell Biol. 2011a;89(2):207-15). The receptor is activated by the three IFN-γ inducible chemokine ligands, namely CXCL9 (also named monokine induced by IFN-γ, MIG), CXCL10 (IFN-γ inducible protein, IP-10) and CXCL11 (IFN-γ inducible T cell a chemoattractant, ITAC) (Groom JR, Luster AD.; Exp Cell Res. 2011b;317(5):620-31). Binding of these chemokines to CXCR3 induces intracellular signaling, leading to T-cell activation and initiation of their recruitment towards sites of inflammation along the gradients of these chemokines (Khan IA, et al.; Immunity. 2000;12(5):483-94; Groom JR, Luster AD.; Exp Cell Res. 2011b;317(5):620-31; Xie JH, et al. J Leukoc Biol. 2003;73(6):771-80). CXCR3 signaling is also involved in T-cell proliferation, polarization, and tissue retention (Alanio C, et al.; J Immunol. 2018;200(1):139-46).

CXCR3 and its ligands are highly upregulated in inflamed tissues of patients with various autoimmune diseases (Steinmetz OM et al.; J Immunol. 2009;183(7):4693-704). As mentioned above, T1D is an autoimmune disease involving the destruction of insulin producing pancreatic islet β cells by autoreactive T-cells, especially CD8+ T cells. Many chemokines, especially those associated with Type 1 T cell responses (Th1, Tc1), like CXCR3 ligands CXCL9 and CXCL10, have been found to be elevated in the serum from patients with T1D in comparison to healthy controls, especially in patients newly diagnosed (Nicoletti F, et al. Diabetologia 2002 45(8):1107-10.; Hakimizadeh E, et al. Clin Lab 2013 59(5-6):531-7). Antonelli et al., demonstrated that the CXCL10 serum levels decline over time in newly diagnosed children with T1D, but are still elevated even 16 months after diagnosis compared to healthy controls (Antonelli A, et al. Cytokine Growth Factor Rev 2014 25(1):57-65). In addition, pancreatic islets from patients with recent onset of T1D (particularly those with remaining functional B cells) have increased levels of both CXCR3 and CXCL10, suggesting that both autoreactive T-cells and the CXCR3/CXCL10 axis have detrimental roles in the development of T1D (Uno S, et al.; Endocr J. 2010;57(11):991-6; Roep BO, et al.; Clin Exp Immunol. 2010;159(3):338-43; Tanaka S, et al. Diabetes. 2009;58(10):2285-91). Uno et al., identified B cells as the main source of CXCL10, and CXCR3 was mainly expressed on T cells in the islet environment (Uno S, et al.; Endocr J. 2010;57(11):991-6). These findings were in line with observations in pancreatic sections from mice with T1D, where CXCL10 was also found to be mainly generated by β-cells and CXCR3 was present on infiltrating leukocytes, including CD8+ T cells (Bender C, et al. Diabetes 2017 66(1):113-26; Carrero JA, et al. PLoS One 2013 8(3):e59701; Sarkar SA, et al. Diabetes 2012 61(2):436-46).

Further, while blockade of CXCL9 with a neutralizing antibody had no influence on the incidence and onset of T1D in earlier experiments, blockade of CXCL10 using neutralizing anti-CXCL10 antibodies or genetic deletion of its receptor CXCR3, significantly delayed T1D in preclinical animal models of T1D (Christen U, et al. J Immunol 2003 171(12):6838-45; Frigerio S, et al. Nature Medicine 2002 8(12):1414-20).

Specifically, in a virus-induced T1D model, preventive administration of an anti-CXCL10 antibody, reduced the incidence of T1D in mice by 70%, associated with reduced insulitis, reduced infiltration of antigen-specific T cells within the islet and maintenance of insulin production (Christen U, et al. J Immunol 2003 171(12):6838-45). However, when this treatment was started later, in already diabetic mice, anti-CXCL10 antibody treatment resulted only in a small non-significant reduction of T1D incidence (Lasch S, et al. Diabetes 2015 64(12):4198-211). In line with these results, Coppieters et al have demonstrated very limited effect of anti-CXCL10 antibody and CXCL10 deficiency on T1D development, in a similar virus-induced T1D model (Coppieters KT, et al. Diabetes 2013 62(7):2492-2499).

Frigerio et al., proposed that CXCR3 antagonists could constitute a promising approach to prevent migration of lymphocytes to the islets of Langerhans, since in CXCR3-deficient mice the onset of T1D was significantly delayed (Frigerio S, et al. Nature Medicine 2002 8(12):1414-20). However, these results were challenged by others showing that CXCR3 deficiency (Coppieters KT, et al. Diabetes 2013 62(7):2492-2499) and administration of a small molecule CXCR3 antagonist (NIBR2130) had subtle or no impact on the development of the disease in a virus-induced T1D model (Christen S, et al. Clin Exp Immunol 2011 165(3):318-28).

Taken together, these variable efficacy results suggest that exclusive blockade of the CXCR3/CXCL10 axis may not be sufficient to inhibit the destructive autoimmune process in

T1D. Therefore, it was hypothesized that neutralization of this chemokine axis may be better suited as part of a combination therapy, such as with an anti-CD3 mAb, which would lead to partial T cell depletion and consequently to an immune reset (Christen U and Kimmel R. Front Endocrinol (Lausanne) 2020 11:591083, doi.org/10.3389/fendo.2020.591083).

It was further hypothesized that combining anti-CD3 mAb treatment—leading to the destruction of aggressive T cells in the islets—with agents blocking the CXCL10/CXCR3 axis, might prevent the re-infiltration of auto-reactive T cells into the islets, and consequently enhance the duration and magnitude of the therapeutic effect afforded by anti-CD3 mAb therapy alone. Indeed, adding a CXCL10 neutralizing monoclonal antibody right after anti-CD3 mAb treatment showed enhanced efficacy in two different T1D mouse models compared to anti-CD3 antibody treatment alone. Importantly, the remission observed was long lasting and none of the cured mice developed T1D at the end of the experiment, six months after initiation of the treatment (Lasch S, et al. Diabetes 2015 64(12):4198-211, WO 2015/154795). The combination of a CXCR3 neutralizing antibody with different immunosuppressants including Muromonab, an anti-CD3 antibody, for the prophylaxis or treatment of T1D was mentioned in WO 2013/109974. Further, the combination therapy of CXCL10 neutralization and an immunomodulator such as an anti-CD3 antibody was proposed by Shigihara et al. (Shigihara T, et al. J Immunology 2005 175(12):8401-08).

CXCL10 neutralizing antibodies have been tested in human clinical trials for autoimmune conditions with mixed results. For example, the CXCL10 neutralizing antibody eldelumab showed positive phase II data in patients with rheumatoid arthritis (Yellin M, et al. Arthritis Rheum 2012 64(6):1730-9), but trends of efficacy in trials for inflammatory bowel disease were only appreciated when patients were stratified for high exposure of the antibody in circulation (Sandborn WJ, et al. J Crohns Colitis 2016 10(4):418-28; Sandborn WJ, et al. J Crohns Colitis 2017 11(7):811-9). These data suggest that due to the high concentration and production rate of CXCL10 in inflamed tissues, high doses and frequent administration of anti-CXCL10 antibody are needed to achieve clinical efficacy. Such dosing regimens are unlikely to lead to a commercially viable and clinically successful therapy for chronic diseases, for which long-term or even life-long treatment is required. This hypothesis is supported by an unsuccessful trial using a different CXCL10 neutralizing antibody (NI-0801) in primary biliary cholangitis. The authors of that trial state that ‘the high production rate of CXCL10 makes it difficult to achieve drug levels that lead to sustained neutralization of the chemokine, thus limiting its targetability’ (De Graaf KL, et al. Hepatol Commun 2018 2(5):492-503). As CXCL10 is elevated in the circulation of patients with T1D and is also very highly expressed in the pancreatic islets, CXCL10 neutralizing antibody therapy is unlikely to achieve a meaningful inhibition of the CXCR3 axis over an extended period using an acceptable dosing regimen.

Another approach to inhibit the CXCR3 axis in the clinics is the use of a small molecule CXCR3 receptor antagonist. Ideally, a CXCR3 antagonist shows characteristics of insurmountability, meaning it depresses the maximal response of the natural agonist and this inhibitory effect is not affected by increasing agonist concentration (Neubig RR, et al. Pharmacol Rev 2003 55(4):597-606). Such a CXCR3 antagonist could hence block the CXCR3 axis even in conditions in which high concentrations of CXCL10 are present. Andrews et al., have recently reviewed the small molecule CXCR3 antagonists identified and described in the literature (Andrews SP and Cox RJ. J Med Chem 2016 59(7):2894-917). From more than 15 chemistry classes identified, only one small molecule CXCR3 antagonist had been investigated in clinical trials until Phase lla but failed to demonstrate efficacy as monotherapy in patients with psoriasis, possibly due to variable exposure (Berry K. et al.; Inflamm. Res. 2004 (Suppl.3), S222).

Surprisingly, it has now been found that a combination treatment with COMPOUND and an anti-CD3 monoclonal antibody is not only efficacious in two different T1D mouse models but shows even improved effects compared to the combination of the anti-CD3 antibody with an anti-CXCL10 antibody (Christen U. et al., Clinical and Experimental Immunology, 2023; uxad083, https://doi.org/10.1093/cei/uxad083).

### FIGURES

FIG. 1: Effect of COMPOUND on BAL CXCR3⁺CD8⁺ T cells in an LPS-induced lung inflammation model

FIG. 1 shows the dose-dependent effect of COMPOUND on CXCR3 expressing CD8⁺T cells recruited to the BAL in the LPS-induced lung inflammation model. Mice were treated from Day-3 with different doses of COMPOUND mixed with food (mg of COMPOUND/g of food, x-axis) and challenged with nebulized LPS on day 0. BAL CXCR3⁺CD8⁺ T cells were quantified by flow cytometry 3 days after LPS challenge. Data are expressed as % control food-treated LPS-challenged group (positive control group), which was set to 100% (y-axis). Data are presented as median with interquartile range; n=11-24/group. *p<0.05, **** p<0.0001 vs. control food-treated mice, using Kruskal-Wallis test, followed by Dunn's multiple comparisons test.

FIG. 2: Effect of treatments on blood glucose values over the 84 days-study in the RIP-LCMV-GP mouse model

FIG. 2 shows the effect of anti-CD3 antibody monotherapy, the combination of anti-CD3 antibody with an anti-CXCL10 antibody, and the combination of anti-CD3 antibody with COMPOUND on blood glucose values in the RIP-LCMV-GP mice. Data are represented as the mean values +SEM. n=11-14/group. *p<0.05, **p<0.01, ****p<0.0001 using two ways ANOVA followed by Tukey's multiple comparisons test. The grey area depicts BGVs that represent the diabetic stage.

FIG. 3: Effect of treatments on BGV in NOD mice

FIG. 3 shows the therapeutic effect of COMPOUND monotherapy, anti-CD3 antibody monotherapy, their combination, and the combination of anti-CD3 antibody with anti-CXCL10 antibody on blood glucose values in NOD mice. Treatments were initiated at T1D onset defined as the first measurement with a BGV≥300 mg/dl. Data are represented as the mean values +SEM. n=14-17/group. ***p<0.001, ****p<0.0001 using Mixed-effects analysis followed by Tukey's multiple comparisons test. The grey area depicts BGVs that represent the diabetic stage.

FIG. 4: Effect of treatments on disease progression from treatment initiation in NOD mice

FIG. 4 shows the therapeutic effect of COMPOUND monotherapy, anti-CD3 antibody monotherapy, their combination, and the combination of anti-CD3 antibody with anti-CXCL10 antibody on T1D disease progression in NOD mice. Disease progression is expressed as the percentage of increase of BGV for each mouse at 40 weeks of age as compared to the BGV at treatment initiation, which was set to 100%. Data are represented as the mean values +SEM. n=14-17/group. ***p<0.001 using one-way ANOVA followed by Tukey's multiple comparisons test.

FIG. 5: Effect of treatments on BGV in non-severe diabetic NOD mice at treatment initiation

FIG. 5 shows the therapeutic effect of COMPOUND monotherapy, anti-CD3 antibody monotherapy, their combination, and the combination of anti-CD3 antibody with an anti-CXCL10 antibody on BGVs in non-severe diabetic NOD mice. Non-severe diabetic NOD mice were defined as mice in which treatment was initiated when BGV was between 300 and 400 mg/dL. Data are represented as the mean values +SEM. n=7-11/group. *p<0.05, ***p<0.001, ****p<0.0001 using Mixed-effects analysis followed by Tukey's multiple comparisons test. The grey area depicts BGVs that represent the diabetic stage.

FIG. 6: Effect of treatments on disease progression from treatment initiation in non-severe diabetic NOD mice

FIG. 6 shows the therapeutic effect of COMPOUND monotherapy, anti-CD3 antibody monotherapy, their combination, and the combination of anti-CD3 antibody with an anti-CXCL10 antibody on T1D disease progression in non-severe diabetic NOD mice. Non-severe diabetic NOD mice were defined as mice in which treatment was initiated when BGV was between 300 and 400 mg/dL. Disease progression is expressed as the percentage of increase of BGV for each mouse at 40 weeks of age compared to the BGV at treatment initiation, which was set to 100%. Data are represented as the mean values +SEM. n=7-11/group. *p<0.05, **p<0.01, ***p<0.001 using one-way ANOVA followed by Tukey's multiple comparisons test.

### DESCRIPTION OF THE INVENTION

1) In a first embodiment the present invention relates to a pharmaceutical combination comprising a first active pharmaceutical ingredient which is 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone (hereinafter also referred to as “COMPOUND”), or a pharmaceutically acceptable salt thereof, and a second active pharmaceutical ingredient which is an anti-CD3 monoclonal antibody.

COMPOUND is a potent, insurmountable, and selective CXCR₃ receptor antagonist. COMPOUND has been described to be useful in the prevention/prophylaxis and/or treatment of diseases or disorders that are related to a dysfunction of the CXCR₃ receptor and/or its ligands CXCL9, CXCL10 and CXCL11, such as (auto-)immune/inflammatory mediated disorders; pulmonary disorders; cardiovascular disorders; infectious diseases; fibrotic disorders; neurodegenerative disorders; and tumor diseases; and especially of rheumatoid arthritis, multiple sclerosis, Crohn's disease, ulcerative colitis, systemic lupus erythematosus, lupus nephritis, sarcoidosis, systemic sclerosis, psoriasis, psoriatic arthritis, interstitial cystitis, celiac disease, myasthenia gravis, type 1 diabetes, vitiligo, uveitis, inflammatory myopathies, dry eye disease, thyroiditis including Grave's disease, transplant rejection, acute and/or chronic graft versus host disease, acute lung injury, acute respiratory distress syndrome, asthma, chronic obstructive pulmonary disorder, atherosclerosis, myocarditis, influenza, cerebral malaria, liver cirrhosis, Alzheimer's disease, neurodegeneration, Huntington's chorea, neuromyelitis optica, chronic inflammatory demyelinating polyneuropathy, Guillain-Barre syndrome, brain tumor, colon cancer, breast cancer, and/or metastatic spread of cancer (WO 2016/113344;PCT/EP2022/051786). COMPOUND may be prepared according to the procedure as disclosed in WO 2016/113344.

It is to be understood that the present invention encompasses COMPOUND in any form including amorphous as well as crystalline forms of COMPOUND. It is further to be understood that crystalline forms of COMPOUND encompass all types of crystalline forms of COMPOUND including polymorphs of the mere molecule, solvates and hydrates, molecular salts and co-crystals (when the same molecule can be co-crystallized with different co-crystal formers) provided they are suitable for pharmaceutical administration.
2) A further embodiment of the invention relates to a pharmaceutical combination according to embodiment 1), wherein the first active pharmaceutical ingredient is 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone (i.e. COMPOUND in free, non-salt form).
3) A further embodiment of the invention relates to a pharmaceutical combination according to any one of embodiments 1) or 2), wherein the second active pharmaceutical ingredient is a humanized or fully human anti-CD3 monoclonal antibody.
4) A further embodiment of the invention relates to a pharmaceutical combination according to any one of embodiments 1) or 2), wherein the second active pharmaceutical ingredient is a humanized anti-CD3 monoclonal antibody (especially otelixizumab, teplizumab, or visilizumab).
5) A further embodiment of the invention relates to a pharmaceutical combination according to any one of embodiments 1) or 2), wherein the second active pharmaceutical ingredient is a fully human anti-CD3 monoclonal antibody (especially foralumab).
6) A further embodiment of the invention relates to a pharmaceutical combination according to any one of embodiments 1) or 2), wherein the second active pharmaceutical ingredient is selected from the group consisting of otelixizumab, teplizumab, visilizumab, and foralumab.
7) A further embodiment of the invention relates to a pharmaceutical combination according to any one of embodiments 1) or 2), wherein the second active pharmaceutical ingredient is selected from the group consisting of otelixizumab and teplizumab.
8) A further embodiment of the invention relates to a pharmaceutical combination according to any one of embodiments 1) or 2), wherein the second active pharmaceutical ingredient is selected from the group consisting of teplizumab and foralumab.
9) A further embodiment of the invention relates to a pharmaceutical combination according to any one of embodiments 1) or 2), wherein the second active pharmaceutical ingredient is teplizumab.
10) A further embodiment of the invention relates to a pharmaceutical combination according to any one of embodiments 1) or 2), wherein the second active pharmaceutical ingredient is foralumab.
11) A further embodiment of the invention relates to a pharmaceutical combination according to any one of embodiments 1) or 2), wherein the second active pharmaceutical ingredient is otelixizumab.
12) A further embodiment of the invention relates to a pharmaceutical combination according to any one of embodiments 1) or 2), wherein the second active pharmaceutical ingredient is visilizumab.

Teplizumab is investigated in clinical trials for the prevention or treatment of type 1 diabetes (especially in patients with recent onset type 1 diabetes and in at-risk patients). Teplizumab may be administered by oral, nasal, subcutaneous, or intravenous administration (especially by subcutaneous or intravenous administration; and notably by intravenous administration). A pharmaceutical composition for intravenous infusion typically comprises teplizumab and 0.9% aqueous sodium chloride solution. Teplizumab is not administered chronically but may be administered once daily or every second day (especially once daily) for a treatment period of 6 to 20 days (especially of 10 to 18 days and notably of 12 to 14 days) with or without use of an up-titration regimen (i.e. of a stepwise increase of the daily dose until the target dose is reached); and especially with use of an up-titration regimen. The up-titration may be done by use of 3 to 10 (especially 4 to 6, and notably 5) different doses of teplizumab, wherein the dose at each day of treatment is either equal or higher than the dose at the preceding day and wherein the highest dose of the uptitration is equal to the target dose that is to be administered and/or is administered until the end of the treatment period. Preferably, the dosing scheme consists of administration of a daily increasing dose from day 1 (first day of treatment) until day 5 (i.e., 5 different, consecutively increasing doses) to reach the target dose, and of an unchanged dose, the target dose, between day 5 and end of treatment at day 10 to 18 (notably at day 14). The dose of teplizumab, that is to be administered and/or is administered, may be calculated based on the body surface area (BSA) of the respective patient (measured in square meter [m²]). The cumulative dose of teplizumab may be between 5.0 mg/m² BSA and 15.0 mg/m² BSA (especially between 7.0 mg/m² BSA and 11.0 mg/m² BSA; and notably about 9.0 mg/m² BSA). Alternatively, a cumulative dose of teplizumab may be between 10.0 mg/m² BSA and 12.0 mg/m² BSA. The daily dose (in the absence of an up-titration regimen) or the target dose (in case of an up-titration regimen) of teplizumab may be equal or lower than 1300 ug/m² BSA (especially between 600 μg/m² BSA and 1000 μg/m² BSA; and notably about 826 μg/m² BSA). Alternatively, a daily dose or target dose is about 1030 μg/m² BSA. An example of a preferred dosing scheme is the administration of about 51 μg/m² BSA on day 1 of the treatment, about 103 μg/m² BSA on day 2, about 207 μg/m² BSA on day 3, about 413 μg/m² BSA on day 4, and about 826 μg/m² BSA on each of days 5 to 14. Another example of a preferred dosing scheme is the administration of about 65 μg/m² BSA on day 1 of the treatment, about 125 μg/m² BSA on day 2, about 250 μg/m² BSA on day 3, about 500 μg/m² BSA on day 4, and about 1030 μg/m² BSA on each of days 5 to 14. The treatment with teplizumab may be repeated for one or two (especially one) additional treatment periods, wherein the dosing scheme during the second or third treatment period may be the same or different than the dosing scheme during any earlier treatment period. Preferably, the dosing scheme is the same during different treatment periods. The interval between two treatment periods is at least 5 month (especially 6 to 12 month).

The BSA may be measured or calculated by any formula commonly used for the calculation of body-surface area, especially by the Mosteller formula:

BSA [m²]=((height [cm]×weight [kg])/3600)^{1/2}

Preferably, the BSA is calculated using the Mosteller formula at the first day of treatment of a given treatment period (especially immediately before the first treatment) and is based on the patient's height and weight on that first treatment day.

Foralumab may be administered by oral, nasal, subcutaneous, or intravenous administration (especially by oral, nasal, or subcutaneous administration; and notably by oral, or nasal administration). Foralumab may be administered once daily or every second day (especially once daily) for a treatment period of 3 to 30 days (especially of 4 to 12 days and notably of 5 to 10 days) with or without use of an up-titration regimen (i.e. of a stepwise increase of the daily dose until the target dose is reached); and especially with use of an up-titration regimen. The up-titration may be done by use of 3 to 10 (especially 4 to 6, and notably 5) different doses of foralumab, wherein the dose at each day of treatment is either equal or higher than the dose at the preceding day and wherein the highest dose of the uptitration is equal to the target dose that is to be administered and/or is administered until the end of the treatment period. The daily dose (in the absence of an up-titration regimen) or the target dose (in case of an up-titration regimen) of foralumab may be between 0.1 mg/60 kg body weight of the patient and 10 mg/60 kg body weight of the patient (especially between 0.5 mg/60 kg body weight of the patient and 5.0 mg/60 kg body weight of the patient). Preferred oral dose ranges is 0.1 mg to 5.0 mg daily. Preferred nasal dose ranges is 0.05 mg to 1.0 mg daily. Preferred subcutaneous dose ranges is 0.2 mg to 5.0 mg daily. The treatment with foralumab may be repeated for one or two (especially one) additional treatment periods, wherein the dosing scheme during the second or third treatment period may be the same or different than the dosing scheme during any earlier treatment period. Preferably, the dosing scheme is the same during different treatment periods. Pharmaceutical compositions comprising foralumab for oral, nasal, and subcutaneous administration are described in U.S. Pat. No. 10,688,186.

Otelixizumab may be administered by oral, nasal, subcutaneous, or intravenous administration (especially by intravenous administration). Otelixizumab may be administered once daily or every second day (especially once daily) for a treatment period of 3 to 20 days (especially of 4 to 12 days and notably of 6 to 10 days) with or without use of an up-titration regimen (i.e. of a stepwise increase of the daily dose until the target dose is reached). The up-titration may be done by use of 3 to 10 (especially 4 to 8) different doses of otelixizumab, wherein the dose at each day of treatment is either equal or higher than the dose at the preceding day and wherein the highest dose of the uptitration is equal to the target dose that is to be administered and/or is administered until the end of the treatment period. The daily dose (in the absence of an up-titration regimen) or the target dose (in case of an up-titration regimen) of otelixizumab may be between 0.5 mg and 5.0 mg (especially between 1.0 mg and 3.75 mg; and notably between 1.5 mg and 3.0 mg). The cumulative dose of otelixizumab may be between 4.0 mg and 27.0 mg (especially between 6.0 mg and 18.0 mg; and notably about 9.0 mg). The treatment with otelixizumab may be repeated for one or two (especially one) additional treatment periods, wherein the dosing scheme during the second or third treatment period may be the same or different than the dosing scheme during any earlier treatment period. Preferably, the dosing scheme is the same during different treatment periods.

Visilizumab may be administered by oral, nasal, subcutaneous, or intravenous administration (especially by intravenous administration). Visilizumab may be administered once daily or every second day (especially once daily) for a treatment period of 2 to 10 days (especially of 2 to 5 days and notably of 2 days) with or without use of an up-titration regimen (i.e. of a stepwise increase of the daily dose until the target dose is reached). The up-titration may be done by use of 2 to 5 different doses of visilizumab, wherein the dose at each day of treatment is either equal or higher than the dose at the preceding day and wherein the highest dose of the uptitration is equal to the target dose that is to be administered and/or is administered until the end of the treatment period. The daily dose (in the absence of an up-titration regimen) or the target dose (in case of an up-titration regimen) of visilizumab may be between 3 μg/kg body weight of the patient and 15 μg/kg body weight of the patient (especially between 4 μg/kg body weight of the patient and 12.5 μg/kg body weight of the patient; and notably about 5 μg/kg body weight of the patient). The cumulative dose of visilizumab may be between 6 μg/kg body weight of the patient and 30 μg/kg body weight of the patient (especially between 8 μg/kg body weight of the patient and 25 μg/kg body weight of the patient; and notably about 10 μg/kg body weight of the patient). The treatment with visilizumab may be repeated for one or two (especially one) additional treatment periods, wherein the dosing scheme during the second or third treatment period may be the same or different than the dosing scheme during any earlier treatment period. Preferably, the dosing scheme is the same during different treatment periods.
13) A further embodiment of the invention relates to a pharmaceutical combination according to any one of embodiments 1) to 12), wherein COMPOUND, or a pharmaceutically acceptable salt thereof, is comprised in a pharmaceutical dosage form for oral, or intravenous administration (especially oral administration) of COMPOUND, or of a pharmaceutically acceptable salt thereof.
14) A further embodiment of the invention relates to a pharmaceutical combination according to embodiment 13), wherein COMPOUND is comprised in the pharmaceutical dosage form in a unit dose between 4.0 mg and 100 mg.

In case COMPOUND is administered and/or is to be administered in one unit dose per day (once daily), lower limits of the unit dose of COMPOUND are especially 10 mg, 15 mg and 20 mg, upper limits are 100 mg, 80 mg, and 60 mg. It is to be understood that each lower limit can be combined with each upper limit. Hence all combinations of lower limits and upper limits shall herewith be specifically disclosed. Especially, the unit dose is between 10 mg and 100 mg.

In case COMPOUND is administered and/or is to be administered in two separated unit doses per day (twice daily), lower limits of the unit dose of COMPOUND are especially 4.0 mg, 8.0 mg, 15 mg and 20 mg, upper limits are 50 mg, 40 mg, and 30 mg. It is to be understood that each lower limit can be combined with each upper limit. Hence all combinations of lower limits and upper limits shall herewith be specifically disclosed. Especially, the unit dose is between 8.0 mg and 50 mg; notably, the unit dose is 20 mg.
15) A further embodiment of the invention relates to a pharmaceutical combination according to any one of embodiments 1) to 14), wherein the anti-CD3 monoclonal antibody is comprised in a pharmaceutical dosage form for oral, nasal, subcutaneous, or intravenous administration of the anti-CD3 monoclonal antibody.
16) A further embodiment of the invention relates to a pharmaceutical combination according to any one of embodiments 1) to 14), wherein the anti-CD3 monoclonal antibody is comprised in a pharmaceutical dosage form for intravenous administration of the anti-CD3 monoclonal antibody.
17) A further embodiment of the invention relates to a pharmaceutical combination according to any one of embodiments 1) to 14), wherein the anti-CD3 monoclonal antibody is comprised in a pharmaceutical dosage form for oral, or nasal administration of the anti-CD3 monoclonal antibody.
18) A further embodiment of the invention relates to a pharmaceutical combination according to any one of embodiments 15) to 17), wherein the anti-CD3 monoclonal antibody (especially teplizumab) is comprised in the pharmaceutical dosage form in a unit dose between 50 μg/m2 BSA and 1000 μg/m2 BSA.

Lower limits of the unit dose of the anti-CD3 monoclonal antibody (especially teplizumab) are 50 μg/m² BSA, 100 μg/m² BSA, 400 μg/m² BSA and 600 μg/m² BSA, upper limits are 1000 μg/m² BSA, 900 μg/m² BSA and 826 μg/m² BSA. It is to be understood that each lower limit can be combined with each upper limit. Hence all combinations of lower limits and upper limits shall herewith be specifically disclosed. Especially, the unit dose is about 826 μg/m² BSA. Alternatively, the unit dose is between 800 μg/m² BSA and 1200 μg/m² BSA, and notably about 1030 μg/m² BSA.

The body surface area (BSA) of the respective patient (measured in square meter [m²]) may be measured or calculated by any formula commonly used for the calculation of BSA, especially by the Mosteller formula.
19) A further embodiment of the invention relates to a pharmaceutical combination according to any one of embodiments 15) to 17), wherein the anti-CD3 monoclonal antibody (especially otelixizumab, teplizumab and foralumab) is comprised in the pharmaceutical dosage form in a unit dose between 0.1 mg and 10 mg.

Lower limits of the unit dose of the anti-CD3 monoclonal antibody (especially otelixizumab, teplizumab and foralumab) are 0.1 mg, 0.5 mg, 1.0 mg and 1.5 mg, upper limits are 10 mg, 5.0 mg, 3.0 mg, and 2.0 mg. It is to be understood that each lower limit can be combined with each upper limit. Hence all combinations of lower limits and upper limits shall herewith be specifically disclosed.
20) A further embodiment of the invention relates to a pharmaceutical combination according to any one of embodiments 1) to 19), wherein the first and the second active pharmaceutical ingredient are comprised in a single pharmaceutical composition.

The single pharmaceutical composition comprises as active pharmaceutical ingredients COMPOUND, or a pharmaceutically acceptable salt thereof, and the anti-CD3 monoclonal antibody, and at least one pharmaceutically acceptable carrier material. In the special case of embodiment 20) where one active pharmaceutical ingredient is to be administered and/or is administered more frequently than the other active pharmaceutical ingredient, only one or several (up to the number of administrations for the less frequently administered active pharmaceutical ingredient) of the pharmaceutical compositions needed per day will contain both, the first and the second active pharmaceutical ingredient. For example, where one of the two active pharmaceutical ingredients is administered once daily and the other active pharmaceutical ingredient is administered twice daily, only one of the two pharmaceutical compositions needed per day will contain both, the first and the second active pharmaceutical ingredient whereas the other will only contain the active pharmaceutical ingredient that is administered twice daily.

Moreover, in case of a pharmaceutical combination according to embodiment 20) wherein the first and/or the second active pharmaceutical ingredient is to be administered and/or is admistered according to a dose up-titration regimen, the pharmaceutical compositions needed for the dose up-titration will contain the amounts of active pharmaceutical ingredient required for the different steps of the dose up-titration regimen.
21) A further embodiment of the invention relates to a pharmaceutical combination according to any one of embodiments 1) to 19), wherein the first and the second active pharmaceutical ingredient are comprised in separated pharmaceutical compositions.

One of the separated pharmaceutical compositions comprises as active pharmaceutical ingredient COMPOUND, or a pharmaceutically acceptable salt thereof, and at least one pharmaceutically acceptable carrier material. The other of the separated pharmaceutical compositions comprises as active pharmaceutical ingredient the anti-CD3 monoclonal antibody, and at least one pharmaceutically acceptable carrier material.

In case the first and the second active pharmaceutical ingredient are comprised in separated pharmaceutical compositions, they can be administered simultaneously, sequentially, or separately; preferably the separated pharmaceutical compositions are administered simultaneously or sequentially, especially sequentially. In case the first active pharmaceutical ingredient is for example administered twice daily and the second active pharmaceutical ingredient once daily, then the separated pharmaceutical compositions are preferably administered one time per day simultaneously or sequentially, especially sequentially. If administered sequentially or separately, the separated pharmaceutical compositions may be administered in one or the other order. The number of administrations per day may be the same or different for the separated pharmaceutical compositions. For instance, one pharmaceutical composition may be administered twice daily, and the other pharmaceutical composition may be administered once or twice daily. Preferably the pharmaceutical composition comprising COMPOUND, or a pharmaceutically acceptable salt thereof, is to be administered and/or is administered once or twice daily (especially twice daily) and the pharmaceutical composition comprising the second active pharmaceutical ingredient is to be administered and/or is administered once daily. Further, the separated pharmaceutical compositions may be administered by the same or different routes of administration, preferably by different routes of administration. Most preferably the pharmaceutical composition comprising COMPOUND is to be administered and/or is administered orally, and the pharmaceutical composition comprising the second active pharmaceutical ingredient is to be administered and/or is administered intravenously. The first and/or the second active pharmaceutical ingredient may be independently from each other admistered according to a dose up-titration regimen up to the respective target dose; the pharmaceutical compositions comprising the first and/or the second active pharmaceutical ingredient needed for the dose up-titration will contain the amounts of active pharmaceutical ingredient required for the different steps of the dose up-titration regimen. The two separated pharmaceutical compositions may be administered for the same treatment period or for different treatment periods. Preferably, the pharmaceutical composition comprising COMPOUND, or a pharmaceutically acceptable salt thereof, is to be administered and/or is administered for a longer treatment period (for instance, for more than 30 days, especially for more than one year and notably chronically) than the pharmaceutical composition comprising the second active pharmaceutical ingredient. Preferably, the pharmaceutical composition comprising the second active pharmaceutical ingredient is to be administered and/or is administered for a treatment period of 2 to 30 days. Lower limits of the treatment period for the anti-CD3 monoclonal antibody (especially otelixizumab, teplizumab and foralumab) are 2 days, 4 days, 6 days, and 10 days, upper limits are 30 days, 20 days, 18 days, and 14 days. It is to be understood that each lower limit can be combined with each upper limit. Hence all combinations of lower limits and upper limits shall herewith be specifically disclosed. Most preferably, the treatment period for the anti-CD3 monoclonal antibody (especially teplizumab) is 12 days to 14 days. The first administration of the pharmaceutical composition comprising COMPOUND to a patient may be at the first day of the treatment period with the pharmaceutical composition comprising the second active pharmaceutical ingredient (the anti-CD3 monoclonal antibody); or at any remaining day of said treatment period (especially at day 2 or any other day during the first half of said treatment period); or at any day after the last administration of the pharmaceutical composition comprising the second active pharmaceutical ingredient, i.e. after the end of said treatment period (but not later than 60 days after the end of said treatment period). Preferably, the first administration of the pharmaceutical composition comprising COMPOUND to a patient may be at any day during the treatment period with the pharmaceutical composition comprising the second active pharmaceutical ingredient; or within 30 days (especially 14 days, notably 1 day) thereafter. Most preferably, the first administration of the pharmaceutical composition comprising COMPOUND to a patient may be at the first day of the treatment period with the pharmaceutical composition comprising the second active pharmaceutical ingredient (the anti-CD3 monoclonal antibody).
22) A further embodiment of the invention relates to a pharmaceutical combination according to any one of embodiments 1) to 21) for use as a medicament.
23) A further embodiment of the invention relates to a pharmaceutical combination according to any one of embodiments 1) to 21) for use in the prevention/prophylaxis and/or treatment of a disease or disorder associated with a dysfunction of the CXCR3 receptor and/or a dysfunction of ligands signalling through CXCR3 (CXCL9, CXCL10 and CXCL11).

Such diseases or disorders associated with a dysfunction of the CXCR3 receptor, or its ligands are diseases or disorders where a modulator of a human CXCR3 receptor is required. The above-mentioned diseases or disorders may in particular be defined as comprising (auto-)immune/inflammatory mediated disorders; pulmonary disorders; cardiovascular disorders; infectious diseases; fibrotic disorders; neurodegenerative disorders; and tumor diseases.

(Auto-)immune/inflammatory mediated disorders may be defined as comprising rheumatoid arthritis (RA); multiple sclerosis (MS); inflammatory bowel disease (IBD; comprising Crohn's disease and ulcerative colitis); primary biliary cirrhosis (PBC); autoimmune hepatitis; systemic lupus erythematosus (SLE); lupus nephritis; antiphospholipid syndrome; Sjögren Syndrome; sarcoidosis; systemic sclerosis; spondylarthrites; psoriasis; psoriatic arthritis; interstitial cystitis; celiac disease; thyroiditis such as Hashimoto's thyroiditis, lymphocytic thyroiditis, Grave's disease; myasthenia gravis; type 1 diabetes (especially autoimmune T1D); uveitis; episcleritis; scleritis; Kawasaki's disease; uveo-retinitis; posterior uveitis; uveitis associated with Behcet's disease; uveomeningitis syndrome; vitiligo; allergic encephalomyelitis; atopic diseases such as rhinitis, conjunctivitis, dermatitis; post-infectious autoimmune diseases including rheumatic fever and post-infectious glomerulonephritis; myopathies (comprising inflammatory myopathies); obesity and transplant related disorders. Transplant related disorders may be defined as comprising transplant rejection such as rejection of transplanted organs such as kidney, liver, heart, lung, pancreas, cornea, and skin; acute and/or chronic graft-versus-host diseases; and chronic allograft vasculopathy.

Pulmonary disorders may be defined as comprising acute lung injury; acute respiratory distress syndrome; asthma; and chronic obstructive pulmonary disorder (COPD).

Cardiovascular disorders may be defined as comprising atherosclerosis; and myocarditis.

Infectious diseases may be defined as comprising diseases mediated by various infectious agents and complications resulting threrefrom; such as malaria, cerebral malaria, leprosy, tuberculosis, influenza, toxoplasma gondii, dengue, hepatitis B and C, herpes simplex, leishmania, chlamydia trachomatis, lyme disease, and west nile virus.

Fibrotic disorders may be defined as comprising liver cirrhosis, idiopathic pulmonary fibrosis, renal fibrosis, endomyocardial fibrosis, systemic sclerosis, and arthrofibrosis.

Neurodegenerative disorders may be defined as comprising neurodegeneration and conditions involving neuronal death such as multiple sclerosis (including relapsing remitting multiple sclerosis and progressive multiple sclerosis), Alzheimer's disease, Parkinson's disease, Huntington's chorea, HIV associated dementia, prion mediated neurodegeneration, epilepsy, stroke, cerebral ischemia, cerebral palsy, neuromyelitis optica, clinically isolated syndrome, Alpers' disease, amyotrophic lateral sclerosis (ALS), senile dementia, dementia with Lewy bodies, Rett syndrome, spinal cord trauma, traumatic brain injury, trigeminal neuralgia, chronic inflammatory demyelinating polyneuropathy, Guillain-Barre syndrome, narcolepsy, glossopharyngeal neuralgia, mild cognitive decline, cognitive decline, spinal muscular atrophy, and cerebral malaria. Stroke includes especially intracerebral hemorrhage (ICH); subarachnoid hemorrhage (including aneurysmal subarachnoid hemorrhage); and inflammation and complications related to any of the aforementioned.

Tumor diseases may be defined as comprising all sorts of cancers such as large intestine cancer, rectal cancer, breast cancer, lung cancer, non-small cell lung cancer, prostate cancer, esophagal cancer, stomach cancer, liver cancer, bile duct cancer, spleen cancer, kidney cancer, urinary bladder cancer, uterine cancer, ovarian cancer, cervical cancer, testicular cancer, thyroid cancer, pancreas cancer, brain tumor, blood tumor, basophil adenoma, prolactinoma, hyperprolactinemia, adenomas, endometrial cancer, colon cancer; chronic lymphocytic leukemia (CLL); and (especially) the metastatic spread of cancer.
24) A further embodiment of the invention relates to a pharmaceutical combination according to any one of embodiments 1) to 21) for use in the prevention/prophylaxis and/or treatment of a disease or disorder selected from type 1 diabetes (T1D) (especially autoimmune T1D), multiple sclerosis, vitiligo, organ transplant rejection (especially renal and heart allograft rejection), thyroid eye disease, rheumatoid arthritis, ulcerative colitis, crohn's disease, celiac disease, atherosclerosis, psoriasis, lung inflammation, psoriatic arthritis and hemorrhage (including intracerebral hemorrhage (ICH); subarachnoid hemorrhage (including aneurysmal subarachnoid hemorrhage); and inflammation and complications related to any of the aforementioned).
25) A further embodiment of the invention relates to a pharmaceutical combination according to any one of embodiments 1) to 21) for use in the prevention/prophylaxis and/or treatment of a disease or disorder selected from type 1 diabetes (T1D) (especially autoimmune T1D), multiple sclerosis, vitiligo, organ transplant rejection (especially renal and heart allograft rejection), thyroid eye disease, rheumatoid arthritis, ulcerative colitis, crohn's disease, celiac disease, atherosclerosis, psoriasis, lung inflammation, and psoriatic arthritis.
26) A further embodiment of the invention relates to a pharmaceutical combination according to any one of embodiments 1) to 21) for use in the prevention/prophylaxis and/or treatment of a disease or disorder selected from type 1 diabetes (T1D) (especially autoimmune T1D), multiple sclerosis, organ transplant rejection (especially renal and heart allograft rejection), thyroid eye disease, rheumatoid arthritis, ulcerative colitis, crohn's disease, celiac disease, atherosclerosis, psoriasis, lung inflammation, and psoriatic arthritis.
27) A further embodiment of the invention relates to a pharmaceutical combination according to any one of embodiments 1) to 21) for use in the prevention/prophylaxis and/or treatment of vitiligo.
28) A further embodiment of the invention relates to a pharmaceutical combination according to any one of embodiments 1) to 21) for use in the prevention/prophylaxis and/or treatment of type 1 diabetes (especially autoimmune T1D).

In a first sub-embodiment of embodiment 28), the invention relates to a pharmaceutical combination according to any one of embodiments 1) to 21) for use in the prevention/prophylaxis and/or treatment (especially prevention/prophylaxis) of type 1 diabetes (especially autoimmune T1D) in at-risk patients.

The term “at-risk patient(s)” (or alternatively “at-risk individual(s)”), as used herein in relation to type 1 diabetes, refers to individual(s) having a lifetime risk of developing stage 3 type 1 diabetes of at least 50% (especially at least 65% and notably at least 80%). Especially, “at-risk patient(s)” are individual(s) with two or more T1D-related and/or islet-specific (especially islet specific) autoantibodies. The most important T1D-related and/or islet-specific autoantibodies are anti-glutamic acid decarboxylase 65 (GAD65), anti-islet cell antibody 512 (ICA512), micro-insulin autoantibody (mIAA), zinc transporter 8 (ZnT8) and islet cell antibody (ICA). According to their clinical characteristics, at-risk patients can be classified as stage 1 or stage 2. At-risk patient(s) may be especially defined as individual(s) with two or more T1D-related and/or islet-specific (especially islet specific) autoantibodies who have relatives with T1D (especially first-degree relatives such as siblings, parents, and offsprings, and second-degree relatives such as grandparents, grandchildren, half-siblings, aunts, and uncles; and notably first-degree relatives such as siblings, parents, and offsprings); or as individual(s) with two or more T1D-related and/or islet-specific (especially islet specific) autoantibodies and dysglycemia (diagnosed for instance by a 75 g oral glucose tolerance test (OGTT)); or as individual(s) with two or more T1D-related and/or islet-specific (especially islet specific) autoantibodies and dysglycemia (diagnosed for instance by an OGTT) who have relatives with T1D (especially first-degree relatives such as siblings, parents, and offsprings, and second-degree relatives such as grandparents, grandchildren, half-siblings, aunts, and uncles; and notably first-degree relatives such as siblings, parents, and offsprings). Dysglycemia may be defined by fasting plasma glucose of ≥100 mg/dl (≥5.6 mmol/L) or ≥110 mg/dl (>6.2 mmol/L) (especially by fasting plasma glucose between 100 and 125 mg/dl (≥5.6-6.9 mmol/L), 2-h plasma glucose during OGTT between 140 and 199 mg/dL (7.8 to 11.0 mmol/L), high glucose levels at intermediate time points of an OGTT (30, 60, or 90 min levels of ≥200 mg/dl (>11.1 mmol/L)), and/or HbA_{1c} ≥5.7% (≥39 mmol/mol).

In a second sub-embodiment of embodiment 28), the invention relates to a pharmaceutical combination according to any one of embodiments 1) to 21) for use in the prevention/prophylaxis and/or treatment (especially treatment) of type 1 diabetes (especially autoimmune T1D) in recent onset T1D patients.

The term “recent onset type 1 diabetes (T1D) patient(s)” or “patient(s) with recent onset type 1 diabetes (T1D)”, as used herein, refers to patient(s) who are clinically diagnosed with type 1 diabetes within maximally 150 days (or maximally 105 days; or maximally 56 days; especially maximally 150 days) before treatment initiation (especially before initiation of the treatment with the pharmaceutical combination according to any one of embodiments 1) to 21)). Symptoms and signs of type 1 diabetes may include polyuria, polydipsia, weight loss, asthenia, diabetic ketoacidosis (DKA), and others (especially polyuria, polydipsia, weight loss, asthenia, and diabetic ketoacidosis (DKA)). Patients with recent onset type 1 diabetes require repeated administration of exogenous insulin (even if the administration of exogenous insulin may be interrupted for certain period of times (up to several days or weeks) for a specific patient).

In a third sub-embodiment of embodiment 28), the invention relates to a pharmaceutical combination according to any one of embodiments 1) to 21) for use in the prevention/prophylaxis and/or treatment of type 1 diabetes (especially autoimmune T1D) in at-risk patients and recent onset T1D patients.

The progression of T1D may be described in 3 stages (Insel RA, et al. Diabetes Care 2015 38(10):1964-74):
Stage 1 (asymptomatic stage) represents individuals who have developed two or more type 1 diabetes-associated islet autoantibodies but are normoglycemic;
Stage 2 (pre-symptomatic stage) represents individuals with two or more islet autoantibodies whose disease has progressed to the development of glucose intolerance, or dysglycemia, from loss of functional beta-cell mass;
Stage 3 (symptomatic or clinical stage) represents individuals whose remaining beta-cell capacity is insufficient to maintain glucose control and who require exogenous insulin. Stage 3 represents manifestations of typical clinical symptoms and signs of diabetes, which may include polyuria, polydipsia, weight loss, fatigue, diabetic ketoacidosis (DKA), and others.

The term “prevention/prophylaxis and/or treatment”, as used in the context of the diseases and disorders defined herein, especially refers to the treatment of said diseases and disorders; wherein for progressive diseases and disorders (especially for type 1 diabetes and notably for type 1 diabetes in recent onset T1D patients) the term “treatment” in particular refers to a reduction of the rate of progression of said diseases or disorders (especially of type 1 diabetes and notably of type 1 diabetes in recent onset T1D patients) or to a delay of the worsening of one or more symptoms of said diseases or disorders (especially of type 1 diabetes and notably of type 1 diabetes in recent onset T1D patients). In another aspect the term “prevention/prophylaxis or treatment”, as used in the context of the diseases and disorders defined herein, especially refers to the prevention/prophylaxis of said diseases and disorders (especially of type 1 diabetes and notably of type 1 diabetes in at-risk patients), in particular to preventing or delaying (especially delaying) the onset of said diseases or disorders (especially of type 1 diabetes) in patients who are at risk/are diagnosed as being at risk of developing such disease or disorder (especially type 1 diabetes) or to preventing or delaying (especially delaying) the onset of one or more symptoms of said diseases or disorders (especially of type 1 diabetes and notably of type 1 diabetes in at-risk T1D patients). In the special case of type 1 diabetes (especially of type 1 diabetes in at-risk patients), the term “prevention/prophylaxis”, as used herein, may also refer to the prevention or delay (especially delay) of the transition from an early stage of T1D to a later stage of T1D (such as a transition from stage 1 T1D to stage 2 T1D or from stage 2 T1D to stage 3 T1D; especially from stage 2 T1D to stage 3 T1D).

The present invention also relates to a method for the prevention/prophylaxis and/or treatment of a disease or disorder listed in any one of embodiments 23) to 28) comprising administering to a subject (preferably a human subject) in need thereof a pharmaceutically active amount of a pharmaceutical combination according to any one of embodiments 1) to 21).
29) A further embodiment of the invention relates to a pharmaceutical combination for use according to any one of embodiments 22) to 28), wherein COMPOUND, or a pharmaceutically acceptable salt thereof, (especially COMPOUND) is to be administered and/or is administered to a patient by oral or intravenous administration.
30) A further embodiment of the invention relates to a pharmaceutical combination for use according to any one of embodiments 22) to 28), wherein COMPOUND, or a pharmaceutically acceptable salt thereof, (especially COMPOUND) is to be administered and/or is administered to a patient by oral administration.
31) A further embodiment of the invention relates to a pharmaceutical combination for use according to any one of embodiments 22) to 30), wherein COMPOUND, or a pharmaceutically acceptable salt thereof, (especially COMPOUND) is to be administered and/or is administered to a patient once or twice per day (especially twice daily).
32) A further embodiment of the invention relates to a pharmaceutical combination for use according to embodiment 31), wherein COMPOUND, or a pharmaceutically acceptable salt thereof, (especially COMPOUND) is to be administered and/or is administered to a patient for a treatment period of at least 1 year (especially at least 5 years, and notably at least 10 years).
33) A further embodiment of the invention relates to a pharmaceutical combination for use according to embodiment 31), wherein COMPOUND, or a pharmaceutically acceptable salt thereof, (especially COMPOUND) is to be administered and/or is administered to a patient chronically.

The term “chronical treatment” (or “chronically”) refers to a treatment period starting with the first day of treatment with the respective active pharmaceutical ingredient until a further continuation of the treatment of the patient with the active pharmaceutical ingredient is no longer feasible or indicated. The treatment of the patient with the active pharmaceutical ingredient might be for instance no longer feasible or indicated for reasons such as side effects of the treatment, changes of the health condition of the subject (requiring for instance a different medication), changes of other relevant circumstances in the life of a subject, death of the subject and the like. Especially, the term “chronical treatment” (or “chronically”) refers to a treatment period of at least 10 years, starting with the first day of treatment with the respective active pharmaceutical ingredient until a further continuation of the treatment of the patient with the active pharmaceutical ingredient is not feasible or indicated.
34) A further embodiment of the invention relates to a pharmaceutical combination for use according to any one of embodiments 22) to 33), wherein the amount of COMPOUND, or a pharmaceutically acceptable salt thereof, (especially COMPOUND) that is to be administered and/or is administered to a patient is between 8.0 mg per day and 100 mg per day.

In case COMPOUND is administered and/or is to be administered once daily, lower limits of the amount of COMPOUND that is to be administered and/or is administered to a patient per day are 10 mg, 15 mg, and 20 mg, upper limits are 100 mg, 80 mg, and 60 mg. It is to be understood that each lower limit can be combined with each upper limit. Hence all combinations of lower limits and upper limits shall herewith be specifically disclosed. Especially, the amount of COMPOUND per day is between 10 mg and 100 mg.

In case COMPOUND is administered and/or is to be administered twice daily, lower limits of the amount of COMPOUND that is to be administered and/or is administered to a patient per day are 8.0 mg (especially 2 times 4.0 mg), 16 mg (especially 2 times 8.0 mg), 30 mg (especially 2 times 15 mg), and 40 mg (especially 2 times 20 mg), upper limits are 100 mg (especially 2 times 50 mg), 80 mg (especially 2 times 40 mg), and 60 mg (especially 2 times 30 mg). It is to be understood that each lower limit can be combined with each upper limit. Hence all combinations of lower limits and upper limits shall herewith be specifically disclosed. Especially, the amount of COMPOUND per day is between 16 mg (especially 2 times 8.0 mg) and 100 mg (especially 2 times 50 mg).

For avoidance of doubt, for the present invention any amount/unit dose in mg of COMPOUND refers to the amount/unit dose suitable for the administration of COMPOUND in free base form having a molecular weight of 550.48 g/mol in such amount/unit dose. Such amount/unit dose may need to be adjusted in a pharmaceutical composition in case COMPOUND is present in such composition in a form different from anhydrous free base, such as a in form of a pharmaceutically acceptable salt; and/or a solvate such as a hydrate.
35) A further embodiment of the invention relates to a pharmaceutical combination for use according to any one of embodiments 22) to 34), wherein the anti-CD3 monoclonal antibody (especially otelixizumab, teplizumab, or foralumab) is to be administered and/or is administered to a patient by oral, nasal, subcutaneous, or intravenous administration (especially by oral, nasal, or intravenous administration).
36) A further embodiment of the invention relates to a pharmaceutical combination for use according to any one of embodiments 22) to 34), wherein the anti-CD3 monoclonal antibody (especially otelixizumab, teplizumab, or foralumab) is to be administered and/or is administered to a patient by oral or nasal administration.
37) A further embodiment of the invention relates to a pharmaceutical combination for use according to any one of embodiments 22) to 34), wherein the anti-CD3 monoclonal antibody (especially otelixizumab, teplizumab, or foralumab) is to be administered and/or is administered to a patient by intravenous administration.
38) A further embodiment of the invention relates to a pharmaceutical combination for use according to any one of embodiments 22) to 34), wherein the anti-CD3 monoclonal antibody is teplizumab, and wherein the anti-CD3 monoclonal antibody is to be administered and/or is administered to a patient by intravenous administration.
39) A further embodiment of the invention relates to a pharmaceutical combination for use according to any one of embodiments 22) to 38), wherein the anti-CD3 monoclonal antibody (especially otelixizumab, teplizumab, or foralumab) is to be administered and/or is administered to a patient once or twice per day (especially once daily).
40) A further embodiment of the invention relates to a pharmaceutical combination for use according to any one of embodiments 22) to 38), wherein the anti-CD3 monoclonal antibody is teplizumab, and wherein the anti-CD3 monoclonal antibody is to be administered and/or is administered to a patient once daily.
41) A further embodiment of the invention relates to a pharmaceutical combination for use according to any one of embodiments 22) to 40), wherein the anti-CD3 monoclonal antibody (especially otelixizumab, teplizumab, or foralumab) is to be administered and/or is administered to a patient for a treatment period of 2 to 30 days.

Lower limits of the treatment period for the anti-CD3 monoclonal antibody (especially otelixizumab, teplizumab or foralumab) are 2 days, 4 days, 6 days, and 10 days, upper limits are 30 days, 20 days, 18 days, and 14 days. It is to be understood that each lower limit can be combined with each upper limit. Hence all combinations of lower limits and upper limits shall herewith be specifically disclosed. Most preferably, the treatment period for the anti-CD3 monoclonal antibody (especially teplizumab) is 12 days to 14 days.
42) A further embodiment of the invention relates to a pharmaceutical combination for use according to any one of embodiments 22) to 40), wherein the anti-CD3 monoclonal antibody is teplizumab, and wherein the anti-CD3 monoclonal antibody is to be administered and/or is administered to a patient for a treatment period of 6 to 20 days.

Lower limits of the treatment period for teplizumab are 6 days, 8 days, 10 days, and 12 days, upper limits are 20 days, 18 days, 16 days, and 14 days. It is to be understood that each lower limit can be combined with each upper limit. Hence all combinations of lower limits and upper limits shall herewith be specifically disclosed. Most preferably, the treatment period for teplizumab is 12 days to 14 days.
43) A further embodiment of the invention relates to a pharmaceutical combination for use according to any one of embodiments 22) to 42), wherein the anti-CD3 monoclonal antibody (especially otelixizumab, teplizumab, or foralumab) is to be administered and/or is administered to a patient by use of an up-titration regimen (i.e. by a stepwise increase of the daily dose until the target dose is reached).

The up-titration may be done by use of 3 to 10 (especially 4 to 6, and notably 5) different doses of anti-CD3 monoclonal antibody (especially otelixizumab, teplizumab, or foralumab), wherein the dose at each day of treatment is either equal or higher than the dose at the preceding day and wherein the highest dose of the up-titration is equal to the target dose that is to be administered and/or is administered until the end of the treatment period.

Preferably the dose at each day of treatment during the up-titration is higher than the dose at the preceding day.
44) A further embodiment of the invention relates to a pharmaceutical combination for use according to any one of embodiments 22) to 42), wherein the anti-CD3 monoclonal antibody is teplizumab; and wherein the anti-CD3 monoclonal antibody is to be administered and/or is administered to a patient by use of an up-titration regimen, wherein the dose at each day of treatment is either equal or higher than the dose at the preceding day and wherein the highest dose of the up-titration is equal to the target dose.

Preferably the dose at each day of treatment during the up-titration is higher than the dose at the preceding day.
45) A further embodiment of the invention relates to a pharmaceutical combination for use according to any one of embodiments 22) to 42), wherein the anti-CD3 monoclonal antibody is teplizumab; and wherein the dosing scheme consists of administration of a daily increasing dose from day 1 (first day of treatment) until day 5 (i.e., 5 different, consecutively increasing doses) to reach the target dose, and of an unchanged dose (the target dose) between day 5 and end of treatment at day 10 to 18 (notably at day 12 to 14).
46) A further embodiment of the invention relates to a pharmaceutical combination for use according to any one of embodiments 22) to 42), wherein the anti-CD3 monoclonal antibody is teplizumab; and wherein the dosing scheme consists of administration of about 51 μg/m² BSA on day 1 of the treatment, about 103 μg/m² BSA on day 2, about 207 μg/m² BSA on day 3, about 413 μg/m² BSA on day 4, and about 826 μg/m² BSA on each of days 5 to 14.

Alternatively, the dosing scheme consists of administration of teplizumab of about 65 μg/m² BSA on day 1 of the treatment, about 125 μg/m² BSA on day 2, about 250 μg/m² BSA on day 3, about 500 μg/m² BSA on day 4, and about 1030 μg/m² BSA on each of days 5 to 14.
47) A further embodiment of the invention relates to a pharmaceutical combination for use according to any one of embodiments 22) to 45), wherein the amount of the anti-CD3 monoclonal antibody (especially otelixizumab, teplizumab, or foralumab), that is to be administered and/or is administered to a patient is between 0.1 mg per day and 10 mg per day.

Lower limits of the amount of the anti-CD3 monoclonal antibody (especially otelixizumab, teplizumab and foralumab) that is to be administered and/or is administered to a patient are 0.1 mg per day, 0.5 mg per day, 1.0 mg per day, and 1.5 mg per day, upper limits are 10 mg per day, 5.0 mg per day, 3.0 mg per day, and 2.0 mg per day. It is to be understood that each lower limit can be combined with each upper limit. Hence all combinations of lower limits and upper limits shall herewith be specifically disclosed.
48) A further embodiment of the invention relates to a pharmaceutical combination for use according to any one of embodiments 22) to 45), wherein the anti-CD3 monoclonal antibody is teplizumab; and wherein the amount of the anti-CD3 monoclonal antibody that is to be administered and/or is administered to a patient is between 0.1 mg per day and 4.0 mg per day.

Lower limits of the amount of teplizumab that is to be administered and/or is administered to a patient are 0.1 mg per day, 0.5 mg per day, and 1.0 mg per day, upper limits are 4.0 mg per day, 3.0 mg per day, and 2.0 mg per day. It is to be understood that each lower limit can be combined with each upper limit. Hence all combinations of lower limits and upper limits shall herewith be specifically disclosed.
49) A further embodiment of the invention relates to a pharmaceutical combination for use according to any one of embodiments 22) to 45), wherein the amount of the anti-CD3 monoclonal antibody (especially otelixizumab, teplizumab, or foralumab), that is to be administered and/or is administered to a patient is between 1.5 μg/kg body weight of the patient per day and 200 μg/kg body weight of the patient per day.

Lower limits of the amount of the anti-CD3 monoclonal antibody (especially otelixizumab, teplizumab and foralumab) that is to be administered and/or is administered to a patient are 1.5 μg/kg body weight of the patient per day, 7.5 μg/kg body weight of the patient per day, and 15 μg/kg body weight of the patient per day, upper limits are 200 μg/kg body weight of the patient per day, 100 μg/kg body weight of the patient per day, 60 μg/kg body weight of the patient per day, and 40 μg/kg body weight of the patient per day. It is to be understood that each lower limit can be combined with each upper limit. Hence all combinations of lower limits and upper limits shall herewith be specifically disclosed.
50) A further embodiment of the invention relates to a pharmaceutical combination for use according to any one of embodiments 22) to 45), wherein the anti-CD3 monoclonal antibody is teplizumab; and wherein the amount of the anti-CD3 monoclonal antibody that is to be administered and/or is administered to a patient is between 50 μg/m² BSA per day and 1000 μg/m² BSA per day.

Alternatively, the amount of teplizumab that is to be administered and/or is administered to a patient is between 50 μg/m² BSA per day and 1200 μg/m² BSA per day.

Lower limits of the amount of teplizumab that is to be administered and/or is administered to a patient are 50 μg/m² BSA per day, 100 μg/m² BSA per day, 400 μg/m² BSA per day and 600 μg/m² BSA per day, upper limits are 1200 μg/m² BSA per day, 1030 μg/m² BSA per day, 1000 μg/m² BSA per day, 900 μg/m² BSA per day and 826 μg/m² BSA per day (especially 1000 μg/m² BSA per day, 900 μg/m² BSA per day and 826 μg/m² BSA per day). It is to be understood that each lower limit can be combined with each upper limit. Hence all combinations of lower limits and upper limits shall herewith be specifically disclosed. Especially, the amount of teplizumab that is to be administered and/or is administered to a patient is about 826 μg/m² BSA per day. Alternatively, the amount of teplizumab that is to be administered and/or is administered to a patient is about 1030 μg/m² BSA per day.

The body surface area (BSA) of the respective patient (measured in square meter [m²]) may be measured or calculated by any formula commonly used for the calculation of BSA, especially by the Mosteller formula.
51) A further embodiment of the invention relates to a pharmaceutical combination for use according to any one of embodiments 22) to 50), wherein the cumulative dose of the anti-CD3 monoclonal antibody (especially otelixizumab, teplizumab, or foralumab), that is to be administered and/or is administered to a patient is between 5.0 mg and 40 mg.

Lower limits of the cumulative dose that is to be administered and/or is administered to a patient are 5.0 mg, 7.5 mg, and 10 mg, upper limits are 40 mg, 30 mg, and 25 mg. It is to be understood that each lower limit can be combined with each upper limit. Hence all combinations of lower limits and upper limits shall herewith be specifically disclosed.
52) A further embodiment of the invention relates to a pharmaceutical combination for use according to any one of embodiments 22) to 50), wherein the anti-CD3 monoclonal antibody is teplizumab; and wherein the cumulative dose of the anti-CD3 monoclonal antibody that is to be administered and/or is administered to a patient is between 5.0 mg/m² BSA and 15 mg/m² BSA.

Lower limits of the cumulative dose that is to be administered and/or is administered to a patient are 5.0 mg/m² BSA, 6.5 mg/m² BSA, and 8.0 mg/m² BSA, upper limits are 15 mg/m² BSA, 12 mg/m² BSA, and 10 mg/m² BSA. It is to be understood that each lower limit can be combined with each upper limit. Hence all combinations of lower limits and upper limits 9shall herewith be specifically disclosed. Preferably the cumulative dose of teplizumab that is to be administered and/or is administered to a patient is about 9.0 mg/m² BSA. Alternatively, the cumulative dose of teplizumab that is to be administered and/or is administered to a patient is between 8.0 mg/m² BSA and 12.0 mg/m² BSA.
53) A further embodiment of the invention relates to a pharmaceutical combination for use according to any one of embodiments 22) to 52), wherein the anti-CD3 monoclonal antibody (especially otelixizumab, teplizumab, or foralumab) is to be administered and/or is administered to a patient for one treatment period.
54) A further embodiment of the invention relates to a pharmaceutical combination for use according to any one of embodiments 22) to 52), wherein the anti-CD3 monoclonal antibody (especially otelixizumab, teplizumab, or foralumab; and notably teplizumab) is to be administered and/or is administered to a patient for a first treatment period and for a second treatment period.

It is understood that the dosing scheme and the duration of the treatment period may be the same or different for the first and the second treatment period. Preferably the dosing scheme and the duration of the treatment period are the same for the first and the second treatment period.
55) A further embodiment of the invention relates to a pharmaceutical combination for use according to embodiment 54), wherein the interval between the first and the second treatment period is at least 5 months (especially about 6 to 12 months, notably about 6 months or about 12 months).

For the avoidance of doubt, the interval is calculated from the first day of treatment of the first treatment period until the first day of treatment of the second treatment period. For instance, if the first day of treatment of the first treatment period is 21^{st} February and the first day of treatment of the second treatment period is 21^{st} August of the same year, then the interval is 6 months.
56) A further embodiment of the invention relates to a pharmaceutical combination for use according to any one of embodiments 54) or 55), wherein the anti-CD3 monoclonal antibody (especially otelixizumab, teplizumab, or foralumab; and notably teplizumab) is to be administered and/or is administered to a patient for a third treatment period, wherein the interval between the second and the third treatment period is equal or longer than the interval between the first and the second treatment period.
57) A further embodiment of the invention relates to a pharmaceutical combination for use according to any one of embodiments 22) to 56), wherein the disease is (autoimmune) type 1 diabetes (in patients (individuals) with Stage 2 type 1 diabetes) and wherein the progression from Stage 2 type 1 diabetes to Stage 3 type 1 diabetes is delayed by at least 24 months (especially at least 36 months, and notably at least 48 months) in at least 75% of the patients compared to untreated patients.
58) A further embodiment of the invention relates to a pharmaceutical combination for use according to any one of embodiments 22) to 56), wherein the disease is (autoimmune) type 1 diabetes (in patients (individuals) with Stage 2 type 1 diabetes) and wherein the progression from Stage 2 type 1 diabetes to Stage 3 type 1 diabetes is delayed on average (arithmetic mean) by at least 24 months (especially at least 36 months, and notably at least 48 months) compared to untreated patients (individuals).

The term “untreated patient(s)”, as used herein, refers to patient(s) (individual(s)) not receiving a prevention/prophylaxis or treatment for (autoimmune) type 1 diabetes (especially not receiving a prevention/prophylaxis or treatment that is approved by a health authority, such as the United States Food and Drug Administration (FDA), for the treatment of type 1 diabetes).

The treatment of a patient with Stage 2 type 1 diabetes (especially autoimmune T1D) with a pharmaceutical combination according to any one of embodiments 1) to 21) may delay the progression from Stage 2 type 1 diabetes to Stage 3 type 1 diabetes on average (arithmetic mean) by at least 1 month (especially at least 6 months, and notably at least 12 months) compared to patients treated with the anti-CD3 monoclonal antibody (especially teplizumab) alone.

The treatment of (autoimmune) type 1 diabetes in a patient (especially in a patient with recent onset type 1 diabetes or in an at-risk patient) with a pharmaceutical combination according to any one of embodiments 1) to 21) may delay the decline of the C-peptide level in the patient (especially if compared to an untreated patient and/or a patient treated with the anti-CD3 monoclonal antibody (notably teplizumab) alone).

Further, the treatment of type 1 diabetes (especially autoimmune T1D) in a patient (especially in a patient with recent onset type 1 diabetes or in an at-risk patient) with a pharmaceutical combination according to any one of embodiments 1) to 21) may result one, two and/or three years after the first administration of the anti-CD3 monoclonal antibody (notably teplizumab) in a higher (especially statistically significantly higher) C-peptide level in the patient if compared to the C-peptide level in an untreated patient and/or a patient treated with the anti-CD3 monoclonal antibody (notably teplizumab) alone. For instance, the C-peptide level may be at least 10%, at least 20% or at least 30% higher.

C-peptide is cleaved from pro-insulin during the formation of insulin and is released from the pancreas at the same time and in equal amounts as insulin. The measurement of C-peptide levels can be used to indirectly measure endogeneous insulin production in T1D patients. C-peptide levels can be measured for instance by using a two site immunoenzymometric assay, a two site fluoroimmunometric assay (Greenbaum CJ, et al. Diabetes Care 2008 31:1966-1971 and supplementary material) or by any other method known to a person skilled in the art. Preferably, C-peptide levels are given as area-under-curve (AUC) responses to a 2-hour mixed meal tolerance test (MMTT) or an oral glucose tolerance test (OGTT).

The treatment of type 1 diabetes (especially autoimmune T1D) in a patient (especially in a patient with recent onset type 1 diabetes or in an at-risk patient) with a pharmaceutical combination according to any one of embodiments 1) to 21) may result in a lower exogenous insulin use (especially in a statistically significant lower exogenous insulin use) during at least one, at least two or at least three year(s) after the first administration of the anti-CD3 monoclonal antibody (notably teplizumab) to the patient if compared to the exogenous insulin use in an untreated patient and/or a patient treated with the anti-CD3 monoclonal antibody (notably teplizumab) alone. For instance, the exogenous insulin use may be at least 10%, at least 20% or at least 30% lower.
59) A further embodiment of the invention relates to a pharmaceutical composition comprising (especially containing) as active principle, 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, and at least one therapeutically inert excipient, wherein the pharmaceutical composition is to be administered and/or is administered in combination with a second pharmaceutical composition comprising (especially containing), as active principle, an anti-CD3 monoclonal antibody (especially otelixizumab, teplizumab or foralumab) and at least one therapeutically inert excipient.
60) A further embodiment of the invention relates to a pharmaceutical composition comprising (especially containing) as active principle, 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone and at least one therapeutically inert excipient, wherein the pharmaceutical composition is to be administered and/or is administered in combination with a second pharmaceutical composition comprising (especially containing), as active principle, teplizumab and at least one therapeutically inert excipient.
61) A further embodiment of the invention relates to a pharmaceutical composition according to embodiment 59) or 60) for use as a medicament.
62) A further embodiment of the invention relates to a pharmaceutical composition according to embodiment 59) or 60) for use in the prevention/prophylaxis and/or treatment of a disease or disorder listed in any one of embodiments 23) to 28).
63) A further embodiment of the invention relates to a pharmaceutical composition comprising (especially containing) as active principle, an anti-CD3 monoclonal antibody (especially otelixizumab, teplizumab or foralumab) and at least one therapeutically inert excipient, wherein the pharmaceutical composition is to be administered and/or is administered in combination with a second pharmaceutical composition comprising (especially containing), as active principle, 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, and at least one therapeutically inert excipient.
64) A further embodiment of the invention relates to a pharmaceutical composition comprising (especially containing) as active principle, teplizumab and at least one therapeutically inert excipient, wherein the pharmaceutical composition is to be administered and/or is administered in combination with a second pharmaceutical composition comprising (especially containing), as active principle, 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone and at least one therapeutically inert excipient.
65) A further embodiment of the invention relates to a pharmaceutical composition according to embodiment 63) or 64) for use as a medicament.
66) A further embodiment of the invention relates to a pharmaceutical composition according to embodiment 63) or 64) for use in the prevention/prophylaxis and/or treatment of a disease or disorder listed in any one of embodiments 23) to 28).
67) A further embodiment of the invention relates to a kit of parts comprising a first pharmaceutical composition comprising (especially containing) as active principle, 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, and at least one therapeutically inert excipient; and a second pharmaceutical composition comprising (especially containing), as active principle, an anti-CD3 monoclonal antibody (especially otelixizumab, teplizumab or foralumab) and at least one therapeutically inert excipient.
68) A further embodiment of the invention relates to a kit of parts comprising a first pharmaceutical composition comprising (especially containing) as active principle, 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone and at least one therapeutically inert excipient; and a second pharmaceutical composition comprising (especially containing), as active principle, teplizumab and at least one therapeutically inert excipient.
69) A further embodiment of the invention relates to a kit of parts according to embodiment 67) or 68) further comprising instructions for the simultaneous, sequential or separate administration of the pharmaceutical compositions.
70) A further embodiment of the invention relates to a kit of parts according to any one of embodiments 67) to 69) for use as a medicament.
71) A further embodiment of the invention relates to a kit of parts according to any one of embodiments 67) to 69) for use in the prevention/prophylaxis and/or treatment of a disease or disorder listed in any one of embodiments 23) to 28).
72) A further embodiment of the invention relates to the use of a first active pharmaceutical ingredient which is 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, and of a second active pharmaceutical ingredient which is an anti-CD3 monoclonal antibody (especially otelixizumab, teplizumab or foralumab) for the manufacture of a pharmaceutical composition comprising the first and the second active pharmaceutical ingredient, for use according to any one of embodiments 22) to 58).
73) A further embodiment of the invention relates to the use of a first active pharmaceutical ingredient which is 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, and of a second active pharmaceutical ingredient which is an anti-CD3 monoclonal antibody (especially otelixizumab, teplizumab or foralumab) for the manufacture of two separated pharmaceutical compositions comprising the first active pharmaceutical ingredient in the first pharmaceutical composition and the second active pharmaceutical ingredient in the second pharmaceutical composition, for use according to any one of embodiments 22) to 58).
74) A further embodiment of the invention relates to the use of a first active pharmaceutical ingredient which is 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone and of a second active pharmaceutical ingredient which is teplizumab, for the manufacture of a pharmaceutical composition comprising the first and the second active pharmaceutical ingredient, for use according to any one of embodiments 22) to 58).
75) A further embodiment of the invention relates to the use of a first active pharmaceutical ingredient which is 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone and of a second active pharmaceutical ingredient which is teplizumab for the manufacture of two separated pharmaceutical compositions comprising the first active pharmaceutical ingredient in the first pharmaceutical composition and the second active pharmaceutical ingredient in the second pharmaceutical composition, for use according to any one of embodiments 22) to 58).
76) A further embodiment of the invention relates to the use of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, for the manufacture of a medicament for use, in combination with a second medicament comprising an anti-CD3 monoclonal antibody (especially otelixizumab, teplizumab or foralumab), wherein the use is according to any one of embodiments 22) to 58).
77) A further embodiment of the invention relates to the use of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone for the manufacture of a medicament for use, in combination with a second medicament comprising teplizumab, wherein the use is according to any one of embodiments 22) to 58).
78) A further embodiment of the invention relates to the use of an anti-CD3 monoclonal antibody (especially otelixizumab, teplizumab or foralumab) for the manufacture of a medicament for use, in combination with a second medicament comprising 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, wherein the use is according to any one of embodiments 22) to 58).
79) A further embodiment of the invention relates to the use of teplizumab for the manufacture of a medicament for use, in combination with a second medicament comprising 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, wherein the use is according to any one of embodiments 22) to 58).

It is understood that any embodiment relating to a pharmaceutical combination for use in the prevention/prophylaxis and/or treatment of a disease or disorder as defined herein (especially according to any one of embodiments 23 to 58) also relates
to a method of preventing/prophylaxis of and/or treating said disease or disorder comprising administering to a patient in need thereof an effective amount of COMPOUND, or a pharmaceutically acceptable salt thereof, wherein COMPOUND is administered and/or is to be administered in combination with an effective amount of an anti-CD3 monoclonal antibody (especially otelixizumab, teplizumab or foralumab, notably teplizumab);
to a method of preventing/prophylaxis of and/or treating said disease or disorder comprising administering to a patient in need thereof an effective amount of an anti-CD3 monoclonal antibody (especially otelixizumab, teplizumab or foralumab, notably teplizumab), wherein the anti-CD3 monoclonal antibody is administered and/or is to be administered in combination with an effective amount of COMPOUND, or of a pharmaceutically acceptable salt thereof;
to a method of preventing/prophylaxis of and/or treating said disease or disorder comprising administering to a patient in need thereof an effective amount of a pharmaceutical combination comprising COMPOUND, or a pharmaceutically acceptable salt thereof, and an anti-CD3 monoclonal antibody (especially otelixizumab, teplizumab or foralumab, notably teplizumab).

Based on the dependencies of the different embodiments 1) to 79) as disclosed hereinabove, the following preferred embodiments are thus possible and intended and herewith specifically disclosed in individualised form (other embodiments are also possible and intended based on the dependencies of the different embodiments 1) to 79)): 1, 2+1, 3+1, 3+2+1, 4+2+1, 5+2+1, 6+2+1, 7+2+1, 8+2+1, 9+1, 9+2+1, 10+2+1, 11+2+1, 12+2+1, 13+1, 13+2+1, 13+3+1, 13+3+2+1, 13+4+2+1, 13+5+2+1, 13+6+2+1, 13+7+2+1, 13+8+2+1, 13+9+1, 13+9+2+1, 13+10+2+1, 13+11+2+1, 13+12+2+1, 14+13+1, 14+13+2+1, 14+13+3+1, 14+13+3+2+1, 14+13+4+2+1, 14+13+5+2+1, 14+13+6+2+1, 14+13+7+2+1, 14+13+8+2+1, 14+13+9+1, 14+13+9+2+1, 14+13+10+2+1, 14+13+11+2+1, 14+13+12+2+1, 15+2+1, 15+9+1, 15+14+13+1, 15+9+2+1, 15+14+13+2+1, 15+14+13+3+1, 15+14+13+3+2+1, 15+14+13+4+2+1, 15+14+13+5+2+1, 15+14+13+6+2+1, 15+14+13+7+2+1, 15+14+13+8+2+1, 15+14+13+9+1, 15+14+13+9+2+1, 15+14+13+10+2+1, 15+14+13+11+2+1, 15+14+13+12+2+1, 16+1, 16+2+1, 16+3+1, 16+3+2+1, 16+9+1, 16+9+2+1, 16+10+2+1, 16+14+13+1, 16+14+13+2+1, 16+14+13+3+1, 16+14+13+3+2+1, 16+14+13+4+2+1, 16+14+13+5+2+1, 16+14+13+6+2+1, 16+14+13+7+2+1, 16+14+13+8+2+1, 16+14+13+9+1, 16+14+13+9+2+1, 16+14+13+10+2+1, 16+14+13+11+2+1, 16+14+13+12+2+1, 17+2+1, 17+9+1, 17+9+2+1, 17+14+13+1, 17+14+13+2+1, 17+14+13+3+1, 17+14+13+3+2+1, 17+14+13+4+2+1, 17+14+13+5+2+1, 17+14+13+6+2+1, 17+14+13+7+2+1, 17+14+13+8+2+1, 17+14+13+9+1, 17+14+13+9+2+1, 17+14+13+10+2+1, 17+14+13+11+2+1, 17+14+13+12+2+1, 18+15+2+1, 18+15+9+1, 18+15+9+2+1, 18+15+14+13+1, 18+15+14+13+2+1, 18+15+14+13+3+1, 18+15+14+13+3+2+1, 18+15+14+13+4+2+1, 18+15+14+13+5+2+1, 18+15+14+13+6+2+1, 18+15+14+13+7+2+1, 18+15+14+13+8+2+1, 18+15+14+13+9+1, 18+15+14+13+9+2+1, 18+15+14+13+10+2+1, 18+15+14+13+11+2+1, 18+15+14+13+12+2+1, 18+16+1, 18+16+2+1, 18+16+3+1, 18+16+3+2+1, 18+16+9+1, 18+16+9+2+1, 18+16+10+2+1, 18+16+14+13+1, 18+16+14+13+2+1, 18+16+14+13+3+1, 18+16+14+13+3+2+1, 18+16+14+13+4+2+1, 18+16+14+13+5+2+1, 18+16+14+13+6+2+1, 18+16+14+13+7+2+1, 18+16+14+13+8+2+1, 18+16+14+13+9+1, 18+16+14+13+9+2+1, 18+16+14+13+10+2+1, 18+16+14+13+11+2+1, 18+16+14+13+12+2+1, 18+17+2+1, 18+17+9+1, 18+17+9+2+1, 18+17+14+13+1, 18+17+14+13+2+1, 18+17+14+13+3+1, 18+17+14+13+3+2+1, 18+17+14+13+4+2+1, 18+17+14+13+5+2+1, 18+17+14+13+6+2+1, 18+17+14+13+7+2+1, 18+17+14+13+8+2+1, 18+17+14+13+9+1, 18+17+14+13+9+2+1, 18+17+14+13+10+2+1, 18+17+14+13+11+2+1, 18+17+14+13+12+2+1, 19+15+2+1, 19+15+9+1, 19+15+9+2+1, 19+15+14+13+1, 19+15+14+13+2+1, 19+15+14+13+3+1, 19+15+14+13+3+2+1, 19+15+14+13+4+2+1, 19+15+14+13+5+2+1, 19+15+14+13+6+2+1, 19+15+14+13+7+2+1, 19+15+14+13+8+2+1, 19+15+14+13+9+1, 19+15+14+13+9+2+1, 19+15+14+13+10+2+1, 19+15+14+13+11+2+1, 19+15+14+13+12+2+1, 19+16+1, 19+16+2+1, 19+16+3+1, 19+16+3+2+1, 19+16+9+1, 19+16+9+2+1, 19+16+10+2+1, 19+16+14+13+1, 19+16+14+13+2+1, 19+16+14+13+3+1, 19+16+14+13+3+2+1, 19+16+14+13+4+2+1, 19+16+14+13+5+2+1, 19+16+14+13+6+2+1, 19+16+14+13+7+2+1, 19+16+14+13+8+2+1, 19+16+14+13+9+1, 19+16+14+13+9+2+1, 19+16+14+13+10+2+1, 19+16+14+13+11+2+1, 19+16+14+13+12+2+1, 19+17+2+1, 19+17+9+1, 19+17+9+2+1, 19+17+14+13+1, 19+17+14+13+2+1, 19+17+14+13+3+1, 19+17+14+13+3+2+1, 19+17+14+13+4+2+1, 19+17+14+13+5+2+1, 19+17+14+13+6+2+1, 19+17+14+13+7+2+1, 19+17+14+13+8+2+1, 19+17+14+13+9+1, 19+17+14+13+9+2+1, 19+17+14+13+10+2+1, 19+17+14+13+11+2+1, 19+17+14+13+12+2+1, 20+1, 21+1, 21+2+1, 21+3+1, 21+3+2+1, 21+4+2+1, 21+5+2+1, 21+6+2+1, 21+7+2+1, 21+8+2+1, 21+9+1, 21+9+2+1, 21+10+2+1, 21+11+2+1, 21+12+2+1, 21+13+1, 21+13+2+1, 21+13+3+1, 21+13+3+2+1, 21+13+4+2+1, 21+13+5+2+1, 21+13+6+2+1, 21+13+7+2+1, 21+13+8+2+1, 21+13+9+1, 21+13+9+2+1, 21+13+10+2+1, 21+13+11+2+1, 21+13+12+2+1, 21+14+13+1, 21+14+13+2+1, 21+14+13+3+1, 21+14+13+3+2+1, 21+14+13+4+2+1, 21+14+13+5+2+1, 21+14+13+6+2+1, 21+14+13+7+2+1, 21+14+13+8+2+1, 21+14+13+9+1, 21+14+13+9+2+1, 21+14+13+10+2+1, 21+14+13+11+2+1, 21+14+13+12+2+1, 21+15+2+1, 21+15+9+1, 21+15+9+2+1, 21+15+14+13+1, 21+15+14+13+2+1, 21+15+14+13+3+1, 21+15+14+13+3+2+1, 21+15+14+13+4+2+1, 21+15+14+13+5+2+1, 21+15+14+13+6+2+1, 21+15+14+13+7+2+1, 21+15+14+13+8+2+1, 21+15+14+13+9+1, 21+15+14+13+9+2+1, 21+15+14+13+10+2+1, 21+15+14+13+11+2+1, 21+15+14+13+12+2+1, 21+16+1, 21+16+2+1, 21+16+3+1, 21+16+3+2+1, 21+16+9+1, 21+16+9+2+1, 21+16+10+2+1, 21+16+14+13+1, 21+16+14+13+2+1, 21+16+14+13+3+1, 21+16+14+13+3+2+1, 21+16+14+13+4+2+1, 21+16+14+13+5+2+1, 21+16+14+13+6+2+1, 21+16+14+13+7+2+1, 21+16+14+13+8+2+1, 21+16+14+13+9+1, 21+16+14+13+9+2+1, 21+16+14+13+10+2+1, 21+16+14+13+11+2+1, 21+16+14+13+12+2+1, 21+17+2+1, 21+17+9+1, 21+17+9+2+1, 21+17+14+13+1, 21+17+14+13+2+1, 21+17+14+13+3+1, 21+17+14+13+3+2+1, 21+17+14+13+4+2+1, 21+17+14+13+5+2+1, 21+17+14+13+7+2+1, 21+17+14+13+8+2+1, 21+17+14+13+9+1, 21+17+14+13+6+2+1, 21+17+14+13+9+2+1, 21+17+14+13+10+2+1, 21+17+14+13+11+2+1, 21+17+14+13+12+2+1, 21+18+15+2+1, 21+18+15+9+1, 21+18+15+9+2+1, 21+18+15+14+13+1, 21+18+15+14+13+2+1, 21+18+15+14+13+3+1, 21+18+15+14+13+3+2+1, 21+18+15+14+13+4+2+1, 21+18+15+14+13+5+2+1, 21+18+15+14+13+6+2+1, 21+18+15+14+13+7+2+1, 21+18+15+14+13+8+2+1, 21+18+15+14+13+9+1, 21+18+15+14+13+9+2+1, 21+18+15+14+13+10+2+1, 21+18+15+14+13+11+2+1, 21+18+15+14+13+12+2+1, 21+18+16+1, 21+18+16+2+1, 21+18+16+3+1, 21+18+16+3+2+1, 21+18+16+9+1, 21+18+16+9+2+1, 21+18+16+10+2+1, 21+18+16+14+13+1, 21+18+16+14+13+2+1, 21+18+16+14+13+3+1, 21+18+16+14+13+3+2+1, 21+18+16+14+13+4+2+1, 21+18+16+14+13+5+2+1, 21+18+16+14+13+6+2+1, 21+18+16+14+13+7+2+1, 21+18+16+14+13+8+2+1, 21+18+16+14+13+9+1, 21+18+16+14+13+9+2+1, 21+18+16+14+13+10+2+1, 21+18+16+14+13+11+2+1, 21+18+16+14+13+12+2+1, 21+18+17+2+1, 21+18+17+9+1, 21+18+17+9+2+1, 21+18+17+14+13+1, 21+18+17+14+13+2+1, 21+18+17+14+13+3+1, 21+18+17+14+13+3+2+1, 21+18+17+14+13+4+2+1, 21+18+17+14+13+5+2+1, 21+18+17+14+13+6+2+1, 21+18+17+14+13+7+2+1, 21+18+17+14+13+8+2+1, 21+18+17+14+13+9+1, 21+18+17+14+13+9+2+1, 21+18+17+14+13+10+2+1, 21+18+17+14+13+11+2+1, 21+18+17+14+13+12+2+1, 21+19+15+2+1, 21+19+15+9+1, 21+19+15+9+2+1, 21+19+15+14+13+1, 21+19+15+14+13+2+1, 21+19+15+14+13+3+1, 21+19+15+14+13+3+2+1, 21+19+15+14+13+4+2+1, 21+19+15+14+13+5+2+1, 21+19+15+14+13+6+2+1, 21+19+15+14+13+7+2+1, 21+19+15+14+13+8+2+1, 21+19+15+14+13+9+1, 21+19+15+14+13+9+2+1, 21+19+15+14+13+10+2+1, 21+19+15+14+13+11+2+1, 21+19+15+14+13+12+2+1, 21+19+16+1, 21+19+16+2+1, 21+19+16+3+1, 21+19+16+3+2+1, 21+19+16+9+1, 21+19+16+9+2+1, 21+19+16+10+2+1, 21+19+16+14+13+1, 21+19+16+14+13+2+1, 21+19+16+14+13+3+1, 21+19+16+14+13+3+2+1, 21+19+16+14+13+4+2+1, 21+19+16+14+13+5+2+1, 21+19+16+14+13+6+2+1, 21+19+16+14+13+7+2+1, 21+19+16+14+13+8+2+1, 21+19+16+14+13+9+1, 21+19+16+14+13+9+2+1, 21+19+16+14+13+10+2+1, 21+19+16+14+13+11+2+1, 21+19+16+14+13+12+2+1, 21+19+17+2+1, 21+19+17+9+1, 21+19+17+9+2+1, 21+19+17+14+13+1, 21+19+17+14+13+2+1, 21+19+17+14+13+3+1, 21+19+17+14+13+3+2+1, 21+19+17+14+13+4+2+1, 21+19+17+14+13+5+2+1, 21+19+17+14+13+6+2+1, 21+19+17+14+13+7+2+1, 21+19+17+14+13+8+2+1, 21+19+17+14+13+9+1, 21+19+17+14+13+9+2+1, 21+19+17+14+13+10+2+1, 21+19+17+14+13+11+2+1, 21+19+17+14+13+12+2+1, 22, 23, 24, 25, 26, 27, 28, 29+22, 29+23, 29+26, 29+27, 29+28, 30+22, 30+23, 30+27, 30+28, 31+30+22, 31+30+23, 31+30+27, 31+30+28, 32+31+30+22, 32+31+30+23, 32+31+30+27, 32+31+30+28, 33+31+30+22, 33+31+30+23, 33+31+30+27, 33+31+30+28, 34+30+22, 34+30+23, 34+30+27, 34+30+28, 34+31+30+22, 34+31+30+23, 34+31+30+27, 34+31+30+28, 35+30+22, 35+30+23, 35+30+27, 35+30+28, 35+34+30+22, 35+34+30+23, 35+34+30+27, 35+34+30+28, 35+34+31+30+22, 35+34+31+30+23, 35+34+31+30+27, 35+34+31+30+28, 36+30+22, 36+30+23, 36+30+27, 36+30+28, 36+34+30+22, 36+34+30+23, 36+34+30+27, 36+34+30+28, 36+34+31+30+22, 36+34+31+30+23, 36+34+31+30+27, 36+34+31+30+28, 37+30+22, 37+30+23, 37+30+27, 37+30+28, 37+34+30+22, 37+34+30+23, 37+34+30+27, 37+34+30+28, 37+34+31+30+22, 37+34+31+30+23, 37+34+31+30+27, 37+34+31+30+28, 38+22, 38+23, 38+27, 38+28, 38+30+22, 38+30+23, 38+30+27, 38+30+28, 38+34+30+22, 38+34+30+23, 38+34+30+27, 38+34+30+28, 38+34+31+30+22, 38+34+31+30+23, 38+34+31+30+27, 38+34+31+30+28, 39+30+22, 39+30+23, 39+30+27, 39+30+28, 39+34+30+22, 39+34+30+23, 39+34+30+27, 39+34+30+28, 39+34+31+30+22, 39+34+31+30+23, 39+34+31+30+27, 39+34+31+30+28, 39+38+22, 39+38+23, 39+38+27, 39+38+28, 39+38+30+22, 39+38+30+23, 39+38+30+27, 39+38+30+28, 39+38+34+30+22, 39+38+34+30+23, 39+38+34+30+27, 39+38+34+30+28, 39+38+34+31+30+22, 39+38+34+31+30+23, 39+38+34+31+30+27, 39+38+34+31+30+28, 40+30+22, 40+30+23, 40+30+27, 40+30+28, 40+34+30+22, 40+34+30+23, 40+34+30+27, 40+34+30+28, 40+34+31+30+22, 40+34+31+30+23, 40+34+31+30+27, 40+34+31+30+28, 40+38+27, 40+38+22, 40+38+23, 40+38+28, 40+38+30+22, 40+38+30+23, 40+38+30+27, 40+38+30+28, 40+38+34+30+22, 40+38+34+30+23, 40+38+34+30+27, 40+38+34+30+28, 40+38+34+31+30+22, 40+38+34+31+30+23, 40+38+34+31+30+27, 40+38+34+31+30+28, 41+30+22, 41+30+23, 41+30+27, 41+30+28, 41+34+30+22, 41+34+30+23, 41+34+30+27, 41+34+30+28, 41+34+31+30+22, 41+34+31+30+23, 41+34+31+30+27, 41+34+31+30+28, 41+38+22, 41+38+23, 41+38+27, 41+38+28, 41+38+30+22, 41+38+30+23, 41+38+30+27, 41+38+30+28, 41+38+34+30+22, 41+38+34+30+23, 41+38+34+30+27, 41+38+34+30+28, 41+38+34+31+30+22, 41+38+34+31+30+23, 41+38+34+31+30+27, 41+38+34+31+30+28, 42+22, 42+23, 42+27, 42+28, 42+30+22, 42+30+23, 42+30+27, 42+30+28, 42+34+30+22, 42+34+30+23, 42+34+30+27, 42+34+30+28, 42+34+31+30+22, 42+34+31+30+23, 42+34+31+30+27, 42+34+31+30+28, 42+38+22, 42+38+23, 42+38+27, 42+38+28, 42+38+30+22, 42+38+30+23, 42+38+30+27, 42+38+30+28, 42+38+34+30+22,, 42+38+34+30+23, 42+38+34+30+27, 42+38+34+30+28, 42+38+34+31+30+22, 42+38+34+31+30+23, 42+38+34+31+30+27, 42+38+34+31+30+28, 43+30+22, 43+30+23, 43+30+27, 43+30+28, 43+34+30+22, 43+34+30+23, 43+34+30+27, 43+34+30+28, 43+34+31+30+22, 43+34+31+30+23, 43+34+31+30+27, 43+34+31+30+28, 43+38+22, 43+38+23, 43+38+27, 43+38+28, 43+38+30+22, 43+38+30+23, 43+38+30+27, 43+38+30+28, 43+38+34+30+22, 43+38+34+30+23, 43+38+34+30+27, 43+38+34+30+28, 43+38+34+31+30+22, 43+38+34+31+30+23, 43+38+34+31+30+27, 43+38+34+31+30+28, 44+30+22, 44+30+23, 44+30+27, 44+30+28, 44+34+30+22, 44+34+30+23, 44+34+30+27, 44+34+30+28, 44+34+31+30+22, 44+34+31+30+23, 44+34+31+30+27, 44+34+31+30+28, 44+38+22, 44+38+23, 44+38+27, 44+38+28, 44+38+30+22, 44+38+30+23, 44+38+30+27, 44+38+30+28, 44+38+34+30+22, 44+38+34+30+23, 44+38+34+30+27, 44+38+34+30+28, 44+38+34+31+30+22, 44+38+34+31+30+23, 44+38+34+31+30+27, 44+38+34+31+30+28, 45+22, 45+23, 45+27, 45+28, 45+30+22, 45+30+23, 45+30+27, 45+34+30+22, 45+34+30+23, 45+34+30+27, 45+30+28, 45+34+30+28, 45+34+31+30+22, 45+34+31+30+23, 45+34+31+30+27, 45+34+31+30+28, 45+38+22, 45+38+23, 45+38+27, 45+38+28, 45+38+30+22, 45+38+30+23, 45+38+30+27, 45+38+30+28, 45+38+34+30+22, 45+38+34+30+23, 45+38+34+30+27, 45+38+34+30+28, 45+38+34+31+30+22, 45+38+34+31+30+23, 45+38+34+31+30+27, 45+38+34+31+30+28, 45+42+22, 45+42+23, 45+42+27, 45+42+28, 45+42+30+22, 45+42+30+23, 45+42+30+27, 45+42+30+28, 45+42+34+30+22, 45+42+34+30+23, 45+42+34+30+27, 45+42+34+30+28, 45+42+34+31+30+22, 45+42+34+31+30+23, 45+42+34+31+30+27, 45+42+34+31+30+28, 45+42+38+22, 45+42+38+23, 45+42+38+27, 45+42+38+28, 45+42+38+30+22, 45+42+38+30+23, 45+42+38+30+27, 45+42+38+30+28, 45+42+38+34+30+22, 45+42+38+34+30+23, 45+42+38+34+30+27, 45+42+38+34+30+28, 45+42+38+34+31+30+22, 45+42+38+34+31+30+23, 45+42+38+34+31+30+27, 45+42+38+34+31+30+28, 46+22, 46+23, 46+27, 46+28, 46+30+22, 46+30+23, 46+30+27, 46+30+28, 46+34+30+22, 46+34+30+23, 46+34+30+27, 46+34+30+28, 46+34+31+30+22, 46+34+31+30+23, 46+34+31+30+27, 46+34+31+30+28, 46+38+22, 46+38+23, 46+38+27, 46+38+28, 46+38+30+22, 46+38+30+23, 46+38+30+27, 46+38+30+28, 46+38+34+30+22, 46+38+34+30+23, 46+38+34+30+27, 46+38+34+30+28, 46+38+34+31+30+22, 46+38+34+31+30+23, 46+38+34+31+30+27, 46+38+34+31+30+28, 46+42+22, 46+42+23, 46+42+27, 46+42+28, 46+42+30+22, 46+42+30+23, 46+42+30+27, 46+42+30+28, 46+42+34+30+22, 46+42+34+30+23, 46+42+34+30+27, 46+42+34+30+28, 46+42+34+31+30+22, 46+42+34+31+30+23, 46+42+34+31+30+27, 46+42+34+31+30+28, 46+42+38+22, 46+42+38+23, 46+42+38+27, 46+42+38+28, 46+42+38+30+22, 46+42+38+30+23, 46+42+38+30+27, 46+42+38+30+28, 46+42+38+34+30+22, 46+42+38+34+30+23, 46+42+38+34+30+27, 46+42+38+34+30+28, 46+42+38+34+31+30+22, 46+42+38+34+31+30+23, 46+42+38+34+31+30+27, 46+42+38+34+31+30+28, 47+30+22 47+30+23, 47+30+27, 47+30+28, 47+34+30+22, 47+34+30+23, 47+34+30+27, 47+34+30+28, 47+34+31+30+22, 47+34+31+30+23, 47+34+31+30+27, 47+34+31+30+28, 47+38+22, 47+38+23, 47+38+27, 47+38+28, 47+38+30+22, 47+38+30+23, 47+38+30+27, 47+38+30+28, 47+38+34+30+22, 47+38+34+30+23, 47+38+34+30+27, 47+38+34+30+28, 47+38+34+31+30+22, 47+38+34+31+30+23, 47+38+34+31+30+27, 47+38+34+31+30+28, 48+30+22, 48+30+23, 48+30+27, 48+30+28, 48+34+30+22, 48+34+30+23, 48+34+30+27, 48+34+30+28, 48+34+31+30+22, 48+34+31+30+23, 48+34+31+30+27, 48+34+31+30+28, 48+38+22, 48+38+23, 48+38+27, 48+38+28, 48+38+30+22, 48+38+30+23, 48+38+30+27, 48+38+30+28, 48+38+34+30+22, 48+38+34+30+23, 48+38+34+30+27, 48+38+34+30+28, 48+38+34+31+30+22, 48+38+34+31+30+23, 48+38+34+31+30+27, 48+38+34+31+30+28, 49+30+22, 49+30+23, 49+30+27, 49+30+28, 49+34+30+22, 49+34+30+23, 49+34+30+27, 49+34+30+28, 49+34+31+30+22, 49+34+31+30+23, 49+34+31+30+27, 49+34+31+30+28, 49+38+22, 49+38+23, 49+38+27, 49+38+28, 49+38+30+22, 49+38+30+23, 49+38+30+27, 49+38+30+28, 49+38+34+30+22, 49+38+34+30+23, 49+38+34+30+27, 49+38+34+30+28, 49+38+34+31+30+22, 49+38+34+31+30+23, 49+38+34+31+30+27, 49+38+34+31+30+28, 50+22, 50+23, 50+27, 50+28, 50+30+22, 50+30+23, 50+30+27, 50+30+28, 50+34+30+22, 50+34+30+23, 50+34+30+27, 50+34+30+28, 50+34+31+30+22, 50+34+31+30+23, 50+34+31+30+27, 50+34+31+30+28, 50+38+22, 50+38+23, 50+38+27, 50+38+28, 50+38+30+22, 50+38+30+23, 50+38+30+27, 50+38+30+28, 50+38+34+30+22, 50+38+34+30+23, 50+38+34+30+27, 50+38+34+30+28, 50+38+34+31+30+22, 50+38+34+31+30+23, 50+38+34+31+30+27, 50+38+34+31+30+28, 50+42+22, 50+42+23, 50+42+27, 50+42+28, 50+42+30+22, 50+42+30+23, 50+42+30+27, 50+42+30+28, 50+42+34+30+22, 50+42+34+30+23, 50+42+34+30+27, 50+42+34+30+28, 50+42+34+31+30+22, 50+42+34+31+30+23, 50+42+34+31+30+27, 50+42+34+31+30+28, 50+42+38+22, 50+42+38+23, 50+42+38+27, 50+42+38+28, 50+42+38+30+22, 50+42+38+30+23, 50+42+38+30+27, 50+42+38+30+28, 50+42+38+34+30+22, 50+42+38+34+30+23, 50+42+38+34+30+27, 50+42+38+34+30+28, 50+42+38+34+31+30+22, 50+42+38+34+31+30+23, 50+42+38+34+31+30+27, 50+42+38+34+31+30+28, 50+45+22, 50+45+23, 50+45+27, 50+45+28, 50+45+30+22, 50+45+30+23, 50+45+30+27, 50+45+30+28, 50+45+34+30+22, 50+45+34+30+23, 50+45+34+30+27, 50+45+34+30+28, 50+45+34+31+30+22, 50+45+34+31+30+23, 50+45+34+31+30+27, 50+45+34+31+30+28, 50+45+38+22, 50+45+38+23, 50+45+38+27, 50+45+38+28, 50+45+38+30+22, 50+45+38+30+23, 50+45+38+30+27, 50+45+38+30+28, 50+45+38+34+30+22, 50+45+38+34+30+23, 50+45+38+34+30+27, 50+45+38+34+30+28, 50+45+38+34+31+30+22, 50+45+38+34+31+30+23, 50+45+38+34+31+30+27, 50+45+38+34+31+30+28, 50+45+42+22, 50+45+42+23, 50+45+42+27, 50+45+42+28, 50+45+42+30+22, 50+45+42+30+23, 50+45+42+30+27, 50+45+42+30+28, 50+45+42+34+30+22, 50+45+42+34+30+23, 50+45+42+34+30+27, 50+45+42+34+30+28, 50+45+42+34+31+30+22, 50+45+42+34+31+30+23, 50+45+42+34+31+30+27, 50+45+42+34+31+30+28, 50+45+42+38+22, 50+45+42+38+23, 50+45+42+38+27, 50+45+42+38+28, 50+45+42+38+30+22, 50+45+42+38+30+23, 50+45+42+38+30+27, 50+45+42+38+30+28, 50+45+42+38+34+30+22, 50+45+42+38+34+30+23, 50+45+42+38+34+30+27, 50+45+42+38+34+30+28, 50+45+42+38+34+31+30+22, 50+45+42+38+34+31+30+23, 50+45+42+38+34+31+30+27, 50+45+42+38+34+31+30+28, 51+22, 51+23, 51+27, 51+28, 51+30+22, 51+30+23, 51+30+27, 51+30+28, 51+34+30+22, 51+34+30+23, 51+34+30+27, 51+34+30+28, 51+34+31+30+22, 51+34+31+30+23, 51+34+31+30+27, 51+34+31+30+28, 51+38+22, 51+38+23, 51+38+27, 51+38+28, 51+38+30+22, 51+38+30+23, 51+38+30+27, 51+38+30+28, 51+38+34+30+22, 51+38+34+30+23, 51+38+34+30+27, 51+38+34+30+28, 51+38+34+31+30+22, 51+38+34+31+30+23, 51+38+34+31+30+27, 51+38+34+31+30+28, 51+42+22, 51+42+23, 51+42+27, 51+42+28, 51+42+30+22, 51+42+30+23, 51+42+30+27, 51+42+30+28, 51+42+34+30+22, 51+42+34+30+23, 51+42+34+30+27, 51+42+34+30+28, 51+42+34+31+30+22, 51+42+34+31+30+23, 51+42+34+31+30+27, 51+42+34+31+30+28, 51+42+38+22, 51+42+38+23, 51+42+38+27, 51+42+38+28, 51+42+38+30+22, 51+42+38+30+23, 51+42+38+30+27, 51+42+38+30+28, 51+42+38+34+30+22, 51+42+38+34+30+23, 51+42+38+34+30+27, 51+42+38+34+30+28, 51+42+38+34+31+30+22, 51+42+38+34+31+30+23, 51+42+38+34+31+30+27, 51+42+38+34+31+30+28, 51+45+22, 51+45+23, 51+45+27, 51+45+28, 51+45+30+22, 51+45+30+23, 51+45+30+27, 51+45+30+28, 51+45+34+30+22, 51+45+34+30+23, 51+45+34+30+27, 51+45+34+30+28, 51+45+34+31+30+22, 51+45+34+31+30+23, 51+45+34+31+30+27, 51+45+34+31+30+28, 51+45+38+22, 51+45+38+23, 51+45+38+27, 51+45+38+28, 51+45+38+30+22, 51+45+38+30+23, 51+45+38+30+27, 51+45+38+30+28, 51+45+38+34+30+22, 51+45+38+34+30+23, 51+45+38+34+30+27, 51+45+38+34+30+28, 51+45+38+34+31+30+22, 51+45+38+34+31+30+23, 51+45+38+34+31+30+27, 51+45+38+34+31+30+28, 51+45+42+22, 51+45+42+23, 51+45+42+27, 51+45+42+28, 51+45+42+30+22, 51+45+42+30+23, 51+45+42+30+27, 51+45+42+30+28, 51+45+42+34+30+22, 51+45+42+34+30+23, 51+45+42+34+30+27, 51+45+42+34+30+28, 51+45+42+34+31+30+22, 51+45+42+34+31+30+23, 51+45+42+34+31+30+27, 51+45+42+34+31+30+28, 51+45+42+38+22, 51+45+42+38+23, 51+45+42+38+27, 51+45+42+38+28, 51+45+42+38+30+22, 51+45+42+38+30+23, 51+45+42+38+30+27, 51+45+42+38+30+28, 51+45+42+38+34+30+22, 51+45+42+38+34+30+23, 51+45+42+38+34+30+27, 51+45+42+38+34+30+28, 51+45+42+38+34+31+30+22, 51+45+42+38+34+31+30+23, 51+45+42+38+34+31+30+27, 51+45+42+38+34+31+30+28, 52+30+22, 52+30+23, 52+30+27, 52+30+28, 52+34+30+22, 52+34+30+23, 52+34+30+27, 52+34+30+28, 52+34+31+30+22, 52+34+31+30+23, 52+34+31+30+27, 52+34+31+30+28, 52+38+22, 52+38+23, 52+38+27, 52+38+28, 52+38+30+22, 52+38+30+23, 52+38+30+27, 52+38+30+28, 52+38+34+30+22, 52+38+34+30+23, 52+38+34+30+27, 52+38+34+30+28, 52+38+34+31+30+22, 52+38+34+31+30+23, 52+38+34+31+30+27, 52+38+34+31+30+28, 53+30+22, 53+30+23, 53+30+27, 53+30+28, 53+34+30+22, 53+34+30+23, 53+34+30+27, 53+34+30+28, 53+34+31+30+22, 53+34+31+30+23, 53+34+31+30+27, 53+34+31+30+28, 53+38+22, 53+38+23, 53+38+27, 53+38+28, 53+38+30+22, 53+38+30+23, 53+38+30+27, 53+38+30+28, 53+38+34+30+22, 53+38+34+30+23, 53+38+34+30+27, 53+38+34+30+28, 53+38+34+31+30+22, 53+38+34+31+30+23, 53+38+34+31+30+27, 53+38+34+31+30+28, 54+30+22, 54+30+23, 54+30+27, 54+30+28, 54+34+30+22, 54+34+30+23, 54+34+30+27, 54+34+30+28, 54+34+31+30+22, 54+34+31+30+23, 54+34+31+30+27, 54+34+31+30+28, 54+38+22, 54+38+23, 54+38+27, 54+38+28, 54+38+30+22, 54+38+30+23, 54+38+30+27, 54+38+30+28, 54+38+34+30+22, 54+38+34+30+23, 54+38+34+30+27, 54+38+34+30+28, 54+38+34+31+30+22, 54+38+34+31+30+23, 54+38+34+31+30+27, 54+38+34+31+30+28, 55+54+30+22, 55+54+30+23, 55+54+30+27, 55+54+30+28, 55+54+34+30+22, 55+54+34+30+23, 55+54+34+30+27, 55+54+34+30+28, 55+54+34+31+30+22, 55+54+34+31+30+23, 55+54+34+31+30+27, 55+54+34+31+30+28, 55+54+38+22, 55+54+38+23, 55+54+38+27, 55+54+38+28, 55+54+38+30+22, 55+54+38+30+23, 55+54+38+30+27, 55+54+38+30+28, 55+54+38+34+30+22, 55+54+38+34+30+23, 55+54+38+34+30+27, 55+54+38+34+30+28, 55+54+38+34+31+30+22, 55+54+38+34+31+30+23, 55+54+38+34+31+30+27, 55+54+38+34+31+30+28, 56+54+30+22, 56+54+30+23, 56+54+30+27, 56+54+30+28, 56+54+34+30+22, 56+54+34+30+23, 56+54+34+30+27, 56+54+34+30+28, 56+54+34+31+30+22, 56+54+34+31+30+23, 56+54+34+31+30+27, 56+54+34+31+30+28, 56+54+38+22, 56+54+38+23, 56+54+38+27, 56+54+38+28, 56+54+38+30+22, 56+54+38+30+23, 56+54+38+30+27, 56+54+38+30+28, 56+54+38+34+30+22, 56+54+38+34+30+23, 56+54+38+34+30+27, 56+54+38+34+30+28, 56+54+38+34+31+30+22, 56+54+38+34+31+30+23, 56+54+38+34+31+30+27, 56+54+38+34+31+30+28, 56+55+54+30+22, 56+55+54+30+23, 56+55+54+30+27, 56+55+54+30+28, 56+55+54+34+30+22, 56+55+54+34+30+23, 56+55+54+34+30+27, 56+55+54+34+30+28, 56+55+54+34+31+30+22, 56+55+54+34+31+30+23, 56+55+54+34+31+30+27, 56+55+54+34+31+30+28, 56+55+54+38+22, 56+55+54+38+23, 56+55+54+38+27, 56+55+54+38+28, 56+55+54+38+30+22, 56+55+54+38+30+23, 56+55+54+38+30+27, 56+55+54+38+30+28, 56+55+54+38+34+30+22, 56+55+54+38+34+30+23, 56+55+54+38+34+30+27, 56+55+54+38+34+30+28, 56+55+54+38+34+31+30+22, 56+55+54+38+34+31+30+23, 56+55+54+38+34+31+30+27, 56+55+54+38+34+31+30+28, 57+30+22, 57+30+23, 57+30+27, 57+30+28, 57+34+30+22, 57+34+30+23, 57+34+30+27, 57+34+30+28, 57+34+31+30+22, 57+34+31+30+23, 57+34+31+30+27, 57+34+31+30+28, 57+38+22, 57+38+23, 57+38+27, 57+38+28, 57+38+30+22, 57+38+30+23, 57+38+30+27, 57+38+30+28, 57+38+34+30+22, 57+38+34+30+23, 57+38+34+30+27, 57+38+34+30+28, 57+38+34+31+30+22, 57+38+34+31+30+23, 57+38+34+31+30+27, 57+38+34+31+30+28, 58+30+22, 58+30+23, 58+30+27, 58+30+28, 58+34+30+22, 58+34+30+23, 58+34+30+27, 58+34+30+28, 58+34+31+30+22, 58+34+31+30+23, 58+34+31+30+27, 58+34+31+30+28, 58+38+22, 58+38+23, 58+38+27, 58+38+28, 58+38+30+22, 58+38+30+23, 58+38+30+27, 58+38+30+28, 58+38+34+30+27, 58+38+34+30+28, 58+38+34+31+30+22, 58+38+34+30+22, 58+38+34+30+23, 58+38+34+31+30+23, 58+38+34+31+30+27, 58+38+34+31+30+28, 59, 60, 61+59, 61+60, 62+59, 62+60, 63, 64, 65+63, 65+64, 66+63, 66+64, 67, 68, 69+67, 69+68, 70+67, 70+68, 70+69+67, 70+69+68, 71+67, 71+68, 71+69+67, 71+69+68, 72, 73, 74, 75, 76, 77, 78, 79.

In the list above the numbers refer to the embodiments according to their numbering provided hereinabove whereas “+” indicates the dependency from another embodiment. The different individualised embodiments are separated by commas. In other words, “4+2+1” for example refers to embodiment 4) depending on embodiment 2), depending on embodiment 1), i.e. embodiment “4+2+1” corresponds to the pharmaceutical combination of embodiment 1) further limited by the features of the embodiments 2) and 4).

Definitions provided herein are intended to apply uniformly to the subject matter as defined in any one of embodiments 1) to 79), and, mutatis mutandis, throughout the description and the claims unless an otherwise expressly set out definition provides a broader or narrower definition. It is well understood that a definition or preferred definition of a term or expression defines and may replace the respective term or expression independently of (and in combination with) any definition or preferred definition of any or all other terms or expressions as defined herein.

Any reference to an active pharmaceutical ingredient as defined in any one of embodiments 1) to 79) is to be understood as referring also to the pharmaceutically acceptable salts of such active pharmaceutical ingredient, as appropriate and expedient.

The term “pharmaceutically acceptable salts”, as used herein, refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example ‘Handbook of Pharmaceutical Salts. Properties, Selection and Use.’, P. Heinrich Stahl, Camille G. Wermuth (Eds.), Wiley-VCH, 2008 and ‘Pharmaceutical Salts and Co-crystals’, Johan Wouters and Luc Quéré (Eds.), RSC Publishing, 2012.

The term “pharmaceutical combination”, as used herein, refers to a combination of two or more (especially two) different active pharmaceutical ingredients, wherein the active pharmaceutical ingredients are comprised in a single pharmaceutical composition or in separated pharmaceutical compositions.

The term “active pharmaceutical ingredient”, as used herein, refers to a pharmaceutically active component of a pharmaceutical composition. Examples of active pharmaceutical ingredients, as used herein, are in a first group 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone (COMPOUND), or a pharmaceutically acceptable salt thereof, and in a second group an anti-CD3 monoclonal antibody (especially otelixizumab, teplizumab, visilizumab, and/or foralumab; notably teplizumab).

The term “simultaneous” or “simultaneously”, when used in relation to the administration of active pharmaceutical ingredients or of pharmaceutical compositions, means that the administration of a first active pharmaceutical ingredient (or of a first pharmaceutical composition, respectively) is still ongoing when the administration of a second active pharmaceutical ingredient (or of a second pharmaceutical composition, respectively) is started. Especially, the term “simultaneous” or “simultaneously” means that two active pharmaceutical ingredients (or two pharmaceutical compositions, respectively) are administered at the same time, i.e. with the same starting and end time, as is for instance the case for the administration of two active pharmaceutical ingredients comprised in a single pharmaceutical composition.

The term “sequential” or “sequentially”, when used in relation to the administration of active pharmaceutical ingredients or of pharmaceutical compositions, means that the administration of a second active pharmaceutical ingredient (or of a second pharmaceutical composition, respectively) is started less than one hour after the administration of a first active pharmaceutical ingredient (or of a first pharmaceutical composition, respectively) has been finalized.

The term “separate” or “separately”, when used in relation to the administration of active pharmaceutical ingredients or of pharmaceutical compositions, means that the administration of a second active pharmaceutical ingredient (or of a second pharmaceutical composition, respectively) is started one hour or more (and up to about twelve hours, or up to about 24 hours, especially up to about twelve hours) after the last preceding administration of a first active pharmaceutical ingredient (or of a first pharmaceutical composition, respectively) has been finalized.

The expressions “to be administered in combination” or “for use, in combination” mean simultaneous, sequential, or separate administration of active pharmaceutical ingredients or pharmaceutical compositions.

The term “route of administration”, as used herein, refers to the path by which an active pharmaceutical ingredient (e.g. in form of a pharmaceutical composition in a particular dosage form) enters the body. The active pharmaceutical ingredients may be administered by enteral (especially oral) or parenteral administration (including intravenous, subcutaneous, nasal, or topical application, especially intravenous application). Examples of dosage forms which may be used for the administration of the active pharmaceutical ingredients are tablets, capsules, pills, granules, powders, solutions, suspensions, emulsions, injectable aqueous or oily solutions or suspensions, suppositories, creams, gels, ear or eye drops, nasal spray, skin patches, or aerosols. Dosage forms for oral administration, such as tablets, capsules, pills, solutions, or suspensions are preferred. In case the two active pharmaceutical ingredients are comprised in separated pharmaceutical compositions, said separated pharmaceutical compositions may be administered by the same or different routes of administration using the same or different dosage forms (especially by oral administration, notably as tablet or capsule, for COMPOUND and by intravenous administration, notably as solution, for the anti-CD3 monoclonal antibody).

The production of pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing COMPOUND, or a pharmaceutically acceptable salt thereof, and/or the second active pharmaceutical ingredient, optionally in combination with other therapeutically valuable substances, into a galenical administration form together with suitable, non-toxic, inert, therapeutically compatible solid or liquid carrier materials and, if desired, usual pharmaceutical adjuvants.

The term “unit dose”, as used herein, refers to the amount of an active pharmaceutical ingredient that is administered and/or is to be administered to a patient in a single dose. As an example, a unit dose is the amount of an active pharmaceutical ingredient in a tablet or capsule for oral administration in case a single tablet or capsule is administered and/or is to be administered to the patient per administration.

The term “target dose”, as used herein, refers to the final (highest) daily dose of an up-titration regimen.

The term “cumulative dose”, as used herein (especially in relation to anti-CD3 monoclonal antibodies), refers to the total dose that is administered and/or is to be administered to a patient during one treatment period, i.e., the sum of the doses administered at day 1 of the treatment, at day 2, at day 3 until the last day of treatment of the respective treatment period. For example, if the treatment period for the respective anti-CD3 monoclonal antibody is 10 days, then the cumulative dose is the sum of the doses administered during the 10 days of treatment.

The term “treatment period”, as used herein (especially in relation to anti-CD3 monoclonal antibodies), refers to the period between the first day of a treatment and the last day of an uninterrupted treatment with an active pharmaceutical ingredient, a pharmaceutical composition and/or a medicament, wherein a treatment is “uninterrupted” if it is in accordance with a regularly repeating dosing scheme such as, for example, twice daily, once daily or every second day. For instance, if an anti-CD3 monoclonal antibody is administered and/or is to be administered once daily for a treatment period of 14 days, this means that the anti-CD3 monoclonal antibody is administered and/or is to be administered once at day 1 of the treatment, once at day 2, once at day 3, once at day 4, once at day 5, once at day 6, once at day 7, once at day 8, once at day 9, once at day 10, once at day 11, once at day 12, once at day 13, and once at day 14 (and without any treatment with the anti-CD3 monoclonal antibody at day 15); if the treatment with the anti-CD3 monoclonal antibody is resumed at any day after day 15 (for instance 6 or 12 months after day 1), a new treatment period is initiated.

The term “anti-CD3 monoclonal antibody”, as used herein, refers to a monoclonal antibody or an antibody fragment (such as a Fab fragment antibody, a VHH antibody (also named nanobody) or a single-chain variable-fragment (scFv) antibody) (especially a monoclonal antibody) binding to and/or recognizing a human CD3 (cluster of differentiation 3) surface antigen. Especially, the anti-CD3 monoclonal antibody binds to and/or recognizes the epsilon chain of the CD3/TCR (T-cell receptor) complex. It is preferred that the anti-CD3 monoclonal antibody has only reduced (as compared to non-human species origin anti-CD3 monoclonal antibodies such as especially Muromonab-CD3) or no binding affinity to Fc receptors (especially human Fc receptors). The binding to the Fc receptors may be reduced in monoclonal antibodies by introducing one, two or more (especially one or two) mutations into the Fc portion of the antibody.

The term “humanized anti-CD3 monoclonal antibody”, as used herein, refers to an anti-CD3 monoclonal antibody in which parts of the complementary determining region binding to and/or recognizing a human CD3 (cluster of differentiation 3) surface antigen are from a non-human species origin, grafted into a human IgG backbone.

The term “fully human anti-CD3 monoclonal antibody”, as used herein, refers to an anti-CD3 monoclonal antibody consisting of human immunoglobulins derived from human immunoglobulin genes. Fully human anti-CD3 monoclonal antibodies may be produced by transgenic animals that express human immunoglobulins, bacteriophage display of human antigen-binding fragments, yeast display of human immunoglobulins or other methods (and especially by transgenic animals that express human immunoglobulins, bacteriophage display of human antigen-binding fragments or yeast display of human immunoglobulins).

The term “patient”, as used herein, refers to a mammal patient and especially to a human patient.

Unless used regarding temperatures, the term “about” (or alternatively “around”) placed before a numerical value “X” refers in the current application to an interval extending from X minus 10% of X to X plus 10% of X, and preferably to an interval extending from X minus 5% of X to X plus 5% of X. Especially preferred is an interval extending from X minus 1% of X to X plus 1% of X. In the particular case of temperatures, the term “about” (or alternatively “around”) placed before a temperature “Y” refers in the current application to an interval extending from the temperature Y minus 10° C. to Y plus 10° C., and preferably to an interval extending from Y minus 5° C. to Y plus 5° C. The term “room temperature” as used herein refers to a temperature of about 25° C.

Whenever the word “between” (or alternatively “to”) is used to describe a numerical range, it is to be understood that the end points of the indicated range are explicitly included in the range. For example: if a temperature range is described to be between 40° C. and 80° C. (or described to be 40° C. to 80° C.), this means that the end points 40° C. and 80° C. are included in the range; or if a variable is defined as being an integer between 1 and 4 (or 1 to 4), this means that the variable is the integer 1, 2, 3, or 4.

The present invention also includes isotopically labelled, especially ²H (deuterium) labelled active pharmaceutical ingredients, which active pharmaceutical ingredients are identical to the active pharmaceutical ingredients as defined in any one of embodiments 1) to 79) except that one or more atoms have each been replaced by an atom having the same atomic number but an atomic mass different from the atomic mass usually found in nature. Isotopically labelled, especially ²H (deuterium) labelled active pharmaceutical ingredients and pharmaceutically acceptable salts thereof are within the scope of the present invention. Substitution of hydrogen with the heavier isotope ²H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment only one of the two active pharmaceutical ingredients of the pharmaceutical combination is isotopically labelled. In a preferred embodiment of the invention, the active pharmaceutical ingredients are not isotopically labelled, or one active pharmaceutical ingredient is not isotopically labelled and the other active pharmaceutical ingredient is labelled only with one or more deuterium atoms, or both active pharmaceutical ingredients are each labelled only with one or more deuterium atoms. In a most preferred embodiment, the active pharmaceutical ingredients are not isotopically labelled at all. Isotopically labelled active pharmaceutical ingredients may be prepared in analogy to the methods described for the not isotopically labelled active pharmaceutical ingredients but using the appropriate isotopic variation of suitable reagents or starting materials.

### Experimental Part

### Abbreviations

The following abbreviations are used throughout the specification and the examples:

AUC Area under curve

BAL Bronchoalveolar lavage

BGV Blood glucose value

BSA Body surface area

Fig. Figure

h hour(s)

i.p. Intraperitoneal

i.v. Intravenous

LCMV-GP lymphocytic choriomeningitis virus glycoprotein

LPS Lipopolysaccharide

mAb(s) Monoclonal antibody (antibodies)

min Minute(s)

MMTT Mixed meal tolerance test

NOD Non-obese diabetic

n Number of animals

ns Non significant

OGTT Oral glucose tolerance test

q.d. (quaque die): also qd; once daily

RIP Rat insulin promoter

SEM Standard error of the mean

T1D Type 1 diabetes

COMPOUND may be prepared according to the procedure as disclosed in WO 2016/113344.

### Examples of Therapeutic Uses of COMPOUND as a Monotherapy or in Combination with an Anti-CD3 Monoclonal Antibody

Therapeutic effects can be modeled in multiple animal models indicative of diseases and disorders where both CXCR3 expression or its ligands play a pathogenic role and treatment with an anti-CD3 antibody has shown to have some therapeutic effect (especially T1D disease).

### 1) Dose Finding Experiments

The efficacy of COMPOUND on preventing CXCR3-expressing CD8+ T cell migration to the site of inflammation can be determined in a pilot experiment. The goal is to assess the dose-effect relationship of COMPOUND on pulmonary inflammatory T cell infiltration into the bronchoalveolar lavage (BAL), in a mouse model of lipopolysaccharide (LPS)-induced lung inflammation.

Male DBA/1 mice are exposed for 30 minutes to nebulized LPS (day 0).

Groups of 11 to 22 mice are given food mixed with different concentrations of COMPOUND (food admix) starting 3 days prior the LPS challenge and continuing during the whole duration of the experiment. The study consists of seven treatment groups:
1. Control food admix (no COMPOUND), n=22
2. Food admix containing 0.006 mg COMPOUND per g food, n=12
3. Food admix containing 0.02 mg COMPOUND per g food, n=12
4. Food admix containing 0.06/0.07 mg COMPOUND per g food, n=24
5. Food admix containing 0.2 mg COMPOUND per g food, n=12
6. Food admix containing 0.6 mg COMPOUND per g food, n=11
7. Food admix containing 2 mg COMPOUND per g food, n=11

The experiment is terminated at day 3, 72 hours post LPS challenge. BAL is collected and number of T cells expressing CXCR3 quantified by flow cytometry as described in Pouzol et al., Front Pharmacol 2021,12(2991), doi: 10.3389/fphar.2021.748740. Plasma samples are taken for COMPOUND concentration determination.

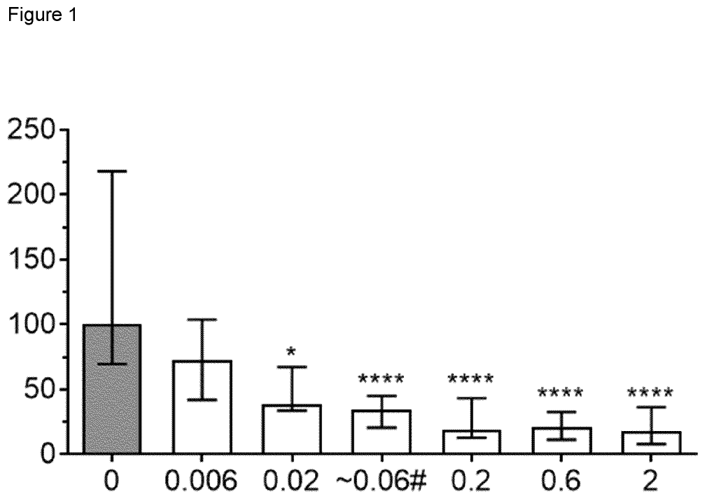

The results from the efficacy experiment are shown in FIG. 1. COMPOUND administered in a preventive setting exhibits dose-dependent efficacy as shown by a dose-dependent decrease of BAL CXCR³⁺CD8⁺ T cells. Efficacy is associated with a dose-dependent increase in COMPOUND plasma concentration.

Based on the pilot experiment described above, one dose of COMPOUND that significantly and maximally reduces CXCR3⁺CD8⁺ T cell migration into the BAL, is selected for the combination efficacy experiment (dose: 0.6 mg of COMPOUND/g of food).

### 2) Combination Efficacy Experiment in a T1D Animal Model

The efficacy of COMPOUND and of an anti-CD3 monoclonal antibody alone or in combination, or the combination of an anti-CD3 monoclonal antibody and anti-CXCL10 monoclonal antibody can be determined in inducible and spontaneous mouse models of T1D. The anti-CD3 (monoclonal armenian hamster anti-mouse CD38 antibody 145-2C11 F (ab') 2 fragment, pepsin digested from BioXCell, reference BE0001-1FAB 4294/0212) and anti-CXCL10 (monoclonal armenian hamster-anti mouse CXCL10 monoclonal antibody (clone 1F11) (Khan et al Immunity 2000, 12:483-494) and their doses selected for the following combination efficacy experiments, have demonstrated efficacy in similar T1D animal models (Lasch S et al. Diabetes 2015, 64:4198-4211). The armenian hamster F (ab') 2 fragment isotype control of the anti-CD3 antibody used as control in the following experiments was obtained from BioXCell, reference BE0091-FAB.

### a) Inducible T1D Model

The first mouse model is an inducible T1D model, in which transgenic C57BL/6 mice express the lymphocytic choriomeningitis virus glycoprotein (LCMV-GP) under control of the rat insulin promoter (RIP) in the β-cells, named RIP-LCMV-GP model (Oldstone MBA et al. Cell 1991, 65:319-331). RIP-LCMV-GP transgenic mice are injected intraperitoneally (i.p.) with LCMV Armstrong clone 53b as previously described (Lasch S et al. Diabetes 2015, 64:4198-4211).

Blood glucose concentrations may be measured using a dynaValeo glucometer from dynamiCARE. RIP-LCMV-GP mice with blood glucose value (BGV)≥300 mg/dl are considered diabetic (Christen U et al. J Immunol 2001, 166:7023-7032).

Groups of 11 to 14 mice that started to develop diabetes between days 10 to 14 after infection are treated. The study consists of four treatment groups:
Group 1: Isotype control i.v., q.d., from day 10 to day 12+ control food from day 1 to day 84 (Isotype/vehicle)
Group 2: Anti-CD3 antibody (3 μg), i.v., q.d., from day 10 to day 12+ control food from day 1 to day 84 ((anti-CD3/vehicle)
Group 3: Anti-CD3 antibody (3 μg), i.v., q.d., from day 10 to day 12+ anti-CXCL10 antibody (100 μg), i.p., from day 13 to day 28 (at days 13, 15, 17, 19, 21, 24, 26, and 28); control food was given from day 1 to 84 (anti-CD3/anti-CXCL10)
Group 4: Anti-CD3 antibody (3 μg), i.v., q.d., from day 10 to day 12+ food mixed with COMPOUND (0.6 mg/g of food) from day 13 to day 84; control food was given from day 1 to 12 (anti-CD3/COMPOUND)

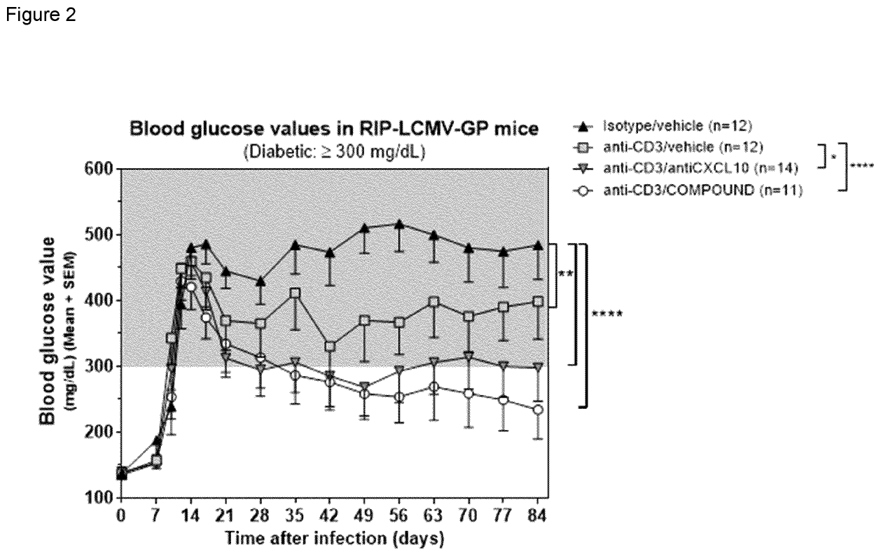

BGVs are monitored on a weekly basis over the entire 12-week-study period and are compared between Isotype/vehicle mice (group 1) and mice receiving either anti-CD3 antibody monotherapy (group 2) or the different combinations with anti-CXCL10 antibody or COMPOUND (groups 3 and 4). The experiment is terminated 84 days after LCMV infection. The results from the combination efficacy experiment are shown in FIG. 2. Overall, both combination therapies were significantly superior to anti-CD3 antibody monotherapy in reducing blood glucose, reaching mean BGV below the diabetic threshold of 300 mg/dL at the end of the study period (297 mg/dl in anti-CD3/anti-CXCL10 group and 234 mg/dl in anti-CD3/COMPOUND group) (FIG. 2).

### b) Spontaneous T1D Model

The efficacy of COMPOUND and the anti-CD3 monoclonal antibody alone or in combination can be determined in non-obese diabetic (NOD) mice, a spontaneous model for studying T1D (Chen et al. Front Endocrinol 2018, 9:51).

Blood glucose concentrations may be measured using a dynaValeo glucometer from dynamiCARE.

NOD mice usually develop diabetes defined as a blood glucose value (BGV)≥300 mg/dl between 13 and 30 weeks of age.

Groups of 14 to 17 mice are treated in a therapeutic setting, starting from the first measurement of diabetes (BGV≥300 mg/dL) for each mouse (day 1). Mice are randomized based on BGV and age at treatment initiation in five treatment groups:
Group 1: Isotype injected i.v., q.d., from day 1 to day 3+ control food from day 1 until end of the study (Isotype/vehicle)
Group 2: Isotype injected i.v., q.d., from day 1 to day 3+ food loaded with COMPOUND (0.6 mg/g of food) from day 1 until end of the study (Isotype/COMPOUND)
Group 3: Anti-CD3 antibody (30 μg), i.v., q.d., from day 1 to day 3+ control food from day 1 until end of the study (anti-CD3/vehicle)
Group 4: Anti-CD3 antibody (30 μg), i.v., q.d., from day 1 to day 3+ anti-CXCL10 antibody (100 μg), i.p., from day 4 to day 19 (administrations every 2^{nd}/3^{rd} day, 8 administations in total)+control food from day 1 until end of the study (anti-CD3/anti-CXCL10)
Group 5: Anti-CD3 antibody (30 μg), i.v., q.d., from day 1 to day 3+ food loaded with COMPOUND (0.6 mg/g of food) from day 1 until end of the study (anti-CD3/COMPOUND)

BGVs are assessed on a weekly basis and are compared between Isotype/control food-treated mice (group 1) and mice receiving the different treatments. For each mouse the experiment is terminated when the respective mouse reaches 40 weeks of age.

This experiment is suitable to show whether the addition of COMPOUND shows added benefit to anti-CD3 antibody monotherapy treatment and whether the combination of anti-CD3 antibody with COMPOUND is superior to the combination of anti-CD3 antibody followed by the anti-CXCL10 antibody.

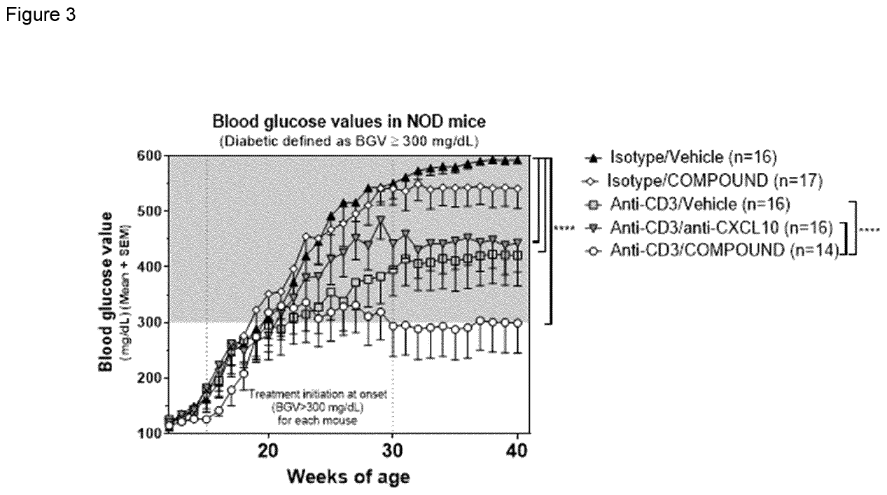

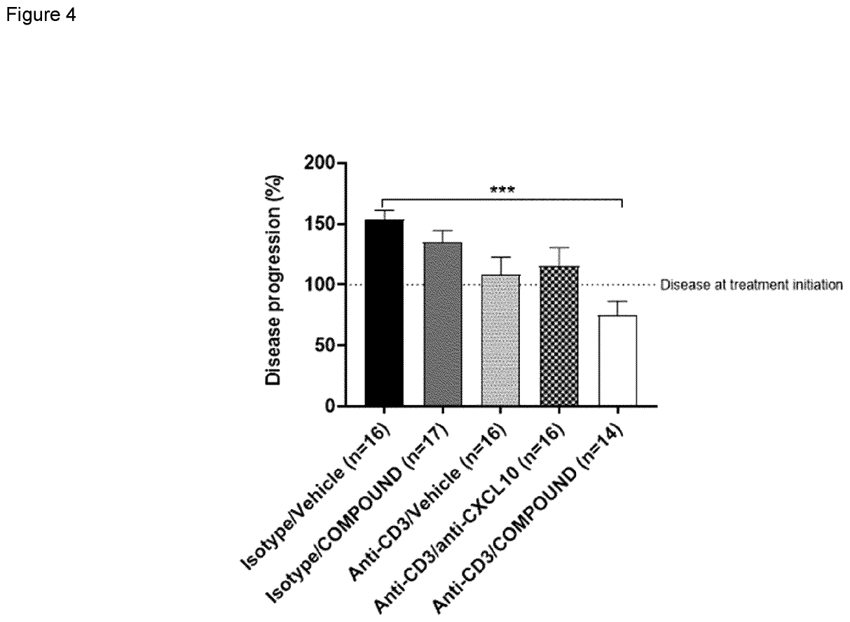

The results from the combination efficacy experiment are shown in FIG. 3 and Table 1. COMPOUND and anti-CD3 antibody administered as monotherapy in a therapeutic setting, exhibited minimal and moderate efficacy, respectively, on the BGV (FIG. 3 and Table 1). When combined, the two treatments showed synergistic efficacy on the BGV (FIG. 3 and Table 1). In addition, only the treatment with the combination of anti-CD3 antibody with COMPOUND significantly reduced and reversed the progression of the disease (defined as a mean BGV <mean BGV at treatment initiation set to 100%) over the study period in contrast to each monotherapy (FIG. 4). The combination of anti-CD3 antibody with anti-CXCL10 antibody was significantly less efficacious than the combination of anti-CD3 antibody with COMPOUND (FIG. 3 and Table 1).

TABLE 1
Effect of treatments on BGVs in NOD mice at week 40 of age
Table 1 shows the therapeutic effect of COMPOUND monotherapy,
anti-CD3 antibody monotherapy, their combination, and the
combination of anti-CD3 antibody with anti-CXCL10 antibody
on BGVs in NOD mice. Data are represented as the mean
values + SEM. n = 14-17/group.

| | BGV at week 40 of | % of reduction |
|---|---|---|
| | age (mg/dL) | from Isotype/ |
| Treatment group | (Mean ± SEM) | vehicle group |
| Group 1: Isotype/vehicle | 592.8 ± 3.2 | — |
| Group 2: Isotype/COMPOUND | 540.9 ± 35.4 | 9 |
| Group 3: anti-CD3/vehicle | 420.8 ± 55.0 | 29 |
| Group 4: anti-CD3/anti-CXCL10 | 443.0 ± 52.2 | 25 |
| Group 5: anti-CD3/COMPOUND | 299.1 ± 54.4 | 50 |

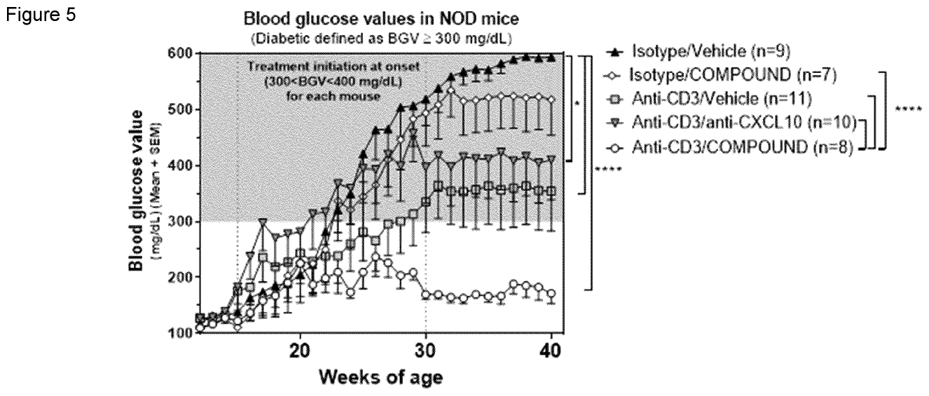

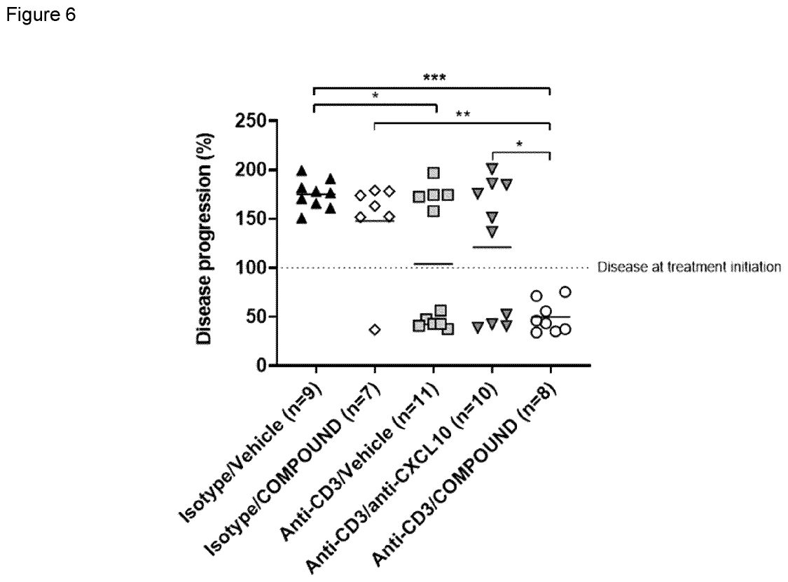

The results from a sub-analysis of the same study are shown in FIG. 5 and Table 2. In this sub-analysis, only the mice not severely hyperglycemic at treatment initiation (defined as 300 mg/dL≤BGV≤400 mg/dL) were included in the study results. COMPOUND and anti-CD3 antibody administered as monotherapy in a therapeutic setting, exhibited minimal and moderate efficacy, respectively, on the BGV (FIG. 5 and Table 2). When combined, the two treatments showed synergistic efficacy on the BGV (FIG. 5 and Table 2). Importantly, T1D was reverted in 100% of the NOD mice treated with the combination of anti-CD3 antibody with COMPOUND in contrast to each monotherapy in which 86% and 45% of the mice treated with COMPOUND or anti-CD3 monotherapy, respectively, showed disease progression, at the end of the study period (FIG. 6). The combination of anti-CD3 antibody with anti-CXCL10 antibody was significantly less efficacious than the combination of anti-CD3 antibody with COMPOUND (FIG. 5 and FIG. 6).

TABLE 2
Effect of treatments on BGVs in non-severe
diabetic NOD mice at week 40 of age
Table 2 shows the therapeutic effect of COMPOUND monotherapy,
anti-CD3 antibody monotherapy, their combination, and the
combination of anti-CD3 antibody with anti-CXCL10 antibody
on BGVs in non-severe diabetic NOD mice. Only the mice
presenting non-severe hyperglycemia (defined as 300
mg/dL ≤ BGV ≤ 400 mg/dL) were included in this sub-study
analysis. Data are represented as the mean values + SEM.
n = 7-11/group.

| | BGV at week 40 of | % of reduction |
|---|---|---|
| | age (mg/dL) | from Isotype/ |
| Treatment group | (Mean ± SEM) | vehicle group |
| Group 1: Isotype/vehicle | 593.2 ± 4.8 | — |
| Group 2: Isotype/COMPOUND | 517.3 ± 63.9 | 13 |
| Group 3: anti-CD3/vehicle | 354.0 ± 71.1 | 40 |
| Group 4: anti-CD3/anti-CXCL10 | 410.0 ± 71.5 | 31 |
| Group 5: anti-CD3/COMPOUND | 171.6 ± 18.4 | 71 |

### 3) Combination Efficacy Experiment in a Vitiligo Animal Model

The efficacy of COMPOUND and of an anti-CD3 monoclonal antibody alone or in combination can be determined in a mouse model of vitiligo based on transient inoculation of B16F10 melanoma cells and depletion of CD4+ regulatory T cells (Chen et al. Cell Regeneration 2022, 11, 31). The anti-CD3 (monoclonal armenian hamster anti-mouse CD3ε antibody 145-2C11 F(ab′)2 fragment, pepsin digested from BioXCell, reference BE0001-1FAB 4294/0212) and COMPOUND and their doses selected for the following combination efficacy experiment, have demonstrated efficacy in T1D animal models (Christen et al. Clinical & Experimental Immunology 2023, doi: 10.1093/cei/uxad083). The armenian hamster F(ab′)2 fragment isotype control of the anti-CD3 antibody used as control in the following experiments was obtained from BioXCell, reference BE0091-FAB. Female C57BL/6 mice are injected with 2×10⁵ B16F10 cells intradermally (day 0). Mice are injected intraperitoneally with anti-CD4 at 200 μg (Clone GK1.5, BioX Cell, Cat BE003-1) on day 4 and day 10. Tumor is removed on days 11 to 12 and mice are injected intraperitoneally with anti-PDL1 antibody at 250 μg (clone 10F.9G2, BioX Cell, cat BE0101) on days 12, 14 and 17.

Mice develop signs of depigmentation which will be graded on a scale from 0 to 6 assessing the extent of hair depigmented defined as: 0=no sign, 1=presence of white hair, 2=at least one patch of white hair>0.5 cm, 3=at least one patch of white hair>2 cm, 4=at least one patch of white hair>3 cm, 5=at least one patch of white hair>3 cm+disseminated white hair and, 6=at least one patch of white hair>3 cm+white hair on large part of the body (>30%). At a cellular level, infiltration of immune cells including CD8+ T cells and melanocyte loss will be monitored by flow cytometry and histology.

Groups of 10-15 mice are treated in a therapeutic setting, starting from day 19. Mice are randomized based on depigmentation status at treatment initiation and on body weight in five treatment groups:
Group 1: healthy mice
Group 2: Isotype injected i.v., q.d., from day 19 to day 21+control food from day 19 until end of the study (isotype/vehicle)
Group 3: Isotype injected i.v., q.d., from day 19 to day 21+food loaded with COMPOUND (0.6 mg/g of food) from day 19 until end of the study (Isotype/COMPOUND)
Group 4: Anti-CD3 antibody (3ug), i.v., q.d., from day 19 to day 21+control food from day 19 until end of the study (anti-CD3/vehicle)
Group 5: Anti-CD3 antibody (3ug), i.v., q.d., from day 19 to day 21+food loaded with COMPOUND (0.6 mg/g of food) from day 19 until end of the study (anti-CD3/COMPOUND)

1. A pharmaceutical combination comprising a first active pharmaceutical ingredient which is 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, and a second active pharmaceutical ingredient which is an anti-CD3 monoclonal antibody.

2. A pharmaceutical combination according to claim 1, wherein the first active pharmaceutical ingredient is 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone.

3. A pharmaceutical combination according to claim 1, wherein the second active pharmaceutical ingredient is a humanized or fully human anti-CD3 monoclonal antibody.

4. A pharmaceutical combination according to claim 1, wherein the second active pharmaceutical ingredient is teplizumab.

5. A pharmaceutical combination according to claim 1, wherein 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, is comprised in a pharmaceutical dosage form for oral or intravenous administration of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or of a pharmaceutically acceptable salt thereof.

6. A pharmaceutical combination according to claim 5, wherein 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone is comprised in the pharmaceutical dosage form in a unit dose between 4.0 mg and 100 mg.

7. A pharmaceutical combination according to claim 1, wherein the anti-CD3 monoclonal antibody is comprised in a pharmaceutical dosage form for intravenous administration of the anti-CD3 monoclonal antibody.

8. A pharmaceutical combination according to claim 7, wherein the anti-CD3 monoclonal antibody is comprised in the pharmaceutical dosage form in a unit dose between 50 μg/m² BSA and 1000 μg/m² BSA.

9. A pharmaceutical combination according to claim 1, wherein the first and the second active pharmaceutical ingredient are comprised in separated pharmaceutical compositions.

10. (canceled)

11. A method for the prevention/prophylaxis and/or treatment of a disease or disorder selected from type 1 diabetes, multiple sclerosis, organ transplant rejection, thyroid eye disease, rheumatoid arthritis, ulcerative colitis, crohn's disease, celiac disease, atherosclerosis, psoriasis, lung inflammation, and psoriatic arthritis in a patient in need thereof, wherein the method comprises administering to the patient an effective amount of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof. and an effective amount of an anti-CD3 monoclonal antibody.

12. A method for the prevention/prophylaxis and/or treatment of type 1 diabetes in a patient in need thereof, wherein the method comprises administering to the patient an effective amount of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2.4]triazol-1-yl)-ethanone, or a pharmaceutically acceptable salt thereof. and an effective amount of an anti-CD3 monoclonal antibody.

13. A method according to claim 11, wherein 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, is administered to the patient once or twice per day.

14. A method according to claim 11, wherein the anti-CD3 monoclonal antibody is administered to the patient for a treatment period of 2 to 30 days.

15. A method according to claim 14, wherein the cumulative dose of the anti-CD3 monoclonal antibody administered to the patient is between 5.0 mg and 40 mg.

16. A method according to claim 12, wherein progression from Stage 2 type 1 diabetes to Stage 3 type 1 diabetes is delayed by at least 24 months in at least 75% of the patients compared to untreated patients.

17. A pharmaceutical composition comprising as active principle 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, and at least one therapeutically inert excipient, wherein the pharmaceutical composition-is to be administered and/or is administered in combination with a second pharmaceutical composition comprising, as active principle, an anti-CD3 monoclonal antibody and at least one therapeutically inert excipient.

18. A method according to claim 12, wherein 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, or a pharmaceutically acceptable salt thereof, is administered to the patient once or twice per day.

19. A method according to claim 12, wherein the anti-CD3 monoclonal antibody is administered to the patient for a treatment period of 2 to 30 days.

20. A method according to claim 19, wherein the cumulative dose of the anti-CD3 monoclonal antibody administered to the patient is between 5.0 mg and 40 mg.

21. A method according to claim 12, wherein the anti-CD3 monoclonal antibody is teplizumab.
